SEQUENCE LISTING (6 sequences)

SEQ ID NO: 1 - DNA, 8622 bp, Homo sapiens
cttgaacccaggaggcagaggttgcagtgagctgagatcgcgccactgtacttcagcctgggtgtcagagcaatactccgttttggaaaacaaacaaacaaacaaacaaacaaaaaacagatggagcaactgagagaggtcttgtgacttgcccaaagtcacacacctcatcactaatcacacctaatcattgagatttggacacacatggttcagttccagagtccatgctccaaaccatgacgacacagtgagagaacattcaaggggagcccagacccagcttcataaccaggcctgtgagcaggagaaagtggaagggatcgtaagtgcccaggggaggcaaagatggactctgcctgaggatctcagagatttcctggaggagggagaattgaggttgggtgttgaaggatgagtgggagttcaccaggaaaagaaggatatggagaaagacattcactcattcaatgaacatctcctgaggacttctgcaagccctgttccgcctggaacggggtgatgctgggacacagagatgagtcagacctgggcccagccctccagaagctgtccacctggtgagaaggaatgatgaggagagaggcagggaggatggggtgatggaagggacaatggggtggggggcagggagatggatgaaaaaaatatatagcaaatgttctcaggatttggcaaagatcaggatgtattaagagagagcacagggcacttgctacctggaaggttgggcacctgggtccttgggtggtggagccgtggggaagggggcaggttatgacaagagtgggttaatccagatggaaccagatttctcaacattctaggagagggccttgtccttgtgggaagaggcccaaatccccagggcagggaaggttctgcaaggtgtgtaaacctgtgcagctgcctgtggtctctgcctcactccacctggatttccctcaatctttcccgtgttctgtctcctcctcccactcctcctctcatcttgggtccttctgtgcctgtacctccctctctttgtatcttttgctcttgtgtctgagtcctgactctgtcttccacccctcgcctcctttctgggtggtccccctgcacatccctccagcctgccgtgggaggttggtctctgcacaccactgctttatccaaaataaacctgctgcaccccaggaccttaggcttcaaggatctccctccttttccaggacacaaaagattctgtatcttgtagcctaaggtgatgaggaatgaggtctcccactctgaagaccccagaggaggtgcccacaacctctccacacccccagcactcctcctccattcagtcaagctctggcccagcaagccgccagttcatcccaaaaggggggtccccctgcacttacctcctctcccaaggcccctgtcacagccccagggcttccccctcccccaggtacatttcccaaccccgattaatcacaggggcggccccatggaggaggaaggagatggcatggcttaccataaagaagcactggacgccgggtgcacgttccaggatccaggtgcccaggggtcatgaagctgggactcctctgtgctctgctctctctgctggcaggtgaggctcccaggctggctgccccttcacggctgtactaaggtcaccttgctcttccctcccatcccaggcttctgcctcctgccctctaggcttctcagcatcctctccctgccctcccagcctgctcttcgctgacccctttgtccctcatccccaccccagggcatggctgggcagacacccgtgccatcggggccgaggaatgtcgccccaactcccagccttggcaggccggcctcttccaccttactcggctcttctgtggggcgaccctcatcagtgaccgctggctgctcacagctgcccactgccgcaagccgtgagtgacccaggctggccatgctggggagggacagaggctgggggtcaggagagggtgaggggtgctttaggccagaagtgcggagcctccacttctgataccacaagttcaactcttagaagtaggaagggtagcctcccaaatcctaaaattctagagaccagcaatatctcatttgagaagtctaagattcgaaacttaggctcttcgaatccgagactgacccagagaaatccagaatcgtagaatcctaaaatcttgaatttatgaaattctgcaatagcctcagcaaattttagaatcatagattcgcagactattagaatcttagcagtctgggtcagcactgcccagaggaattatgatgccagccacatgtgtaagtttaaatttctggtggacacatttaaaaaataaggaatgagtaaaattaattctaatagatttaacttgacatacccaaaaacttattttgacatgtaatcaatttttaaatacgtatgaacgatacagtttacttttgttttggtactaagcctttgaaatctgttctgtattttacacacatagcctgttacaaaatggactagccacatttcaagtgttcaatagccataatggctagtgtgatcctagaatcttaaattcagagctttctagattcattgaatattgaaactcacagtactagaatctttgattcacagtatcctagaatattgagattcagataattctgtagtcttaaactatttgaatcccagactcttaaatttctaaggttatagatttatagaatgatgacattctagtctttcttttttttttttttttttttttttgagacagagtctccctctatctcccaggctggagtgcagtggcacaatctcagctcactgcaacctctgcctctcgggttcaagcaattctcctgcctcagcctcctgagtagctgggattacaggtatgcaccaccatgccaggctattttttttttttttttttttttagtagagacgggggtttcaccatattggccaggctggtcttgaactcctgaccttgtgatctgcccgcctcggcctcccaaagtgctgggattacaggcgtgagccaccgcgcccagccaaaattctagtctttttgtcctagaacattaaaattctatgttcaaatcttagatttaattcagataatgttagaatcctggagtttttttgatccaggggaatctggaatgttagaatcttggattcataaaactctaaaccttgagcctctagattctagaatcatggataatagtgtgtcggaatctgagaattctagaatcttaggttctgggcattctaatagtatcctggaatccacctgatgcaggaatcctctctccattgcctctgaaaagtgaccatccatactgttccaattttcttccctccatgagtaaagcactgattgtggtaagagatgctgtgtgggaatttcccatcatgcattgctccatgatggaacctcctttaacttaagcctatacatcagactgggagaacgatgttcagatttcagccgaaagtgaagcaggagaaatgcagagatatgaaggtggaagagagtgagaggcaggggaagggtagggggatgaagggatgtaggggtgaggactacttttccagatccagagccaagacagcaagaatgacagagagagacagacacagatgtttctggttccccaaccctgaattcgcagtcattagcctgctgcctaatgtcagaggtcagaggctggggaatggacttgtcatccccgaaaggatcccagctgtctagggcatggaccagaaatgaaacaagtgcgctgagactgtggtgagggcttaaggttagacaccaggaagacatgcattgaagggtgaaggatatgatagacaggaaaagctgaggccagagatgacccccaatttggggattttccatatcccatcccctttcatacacacgcacacgtatacacacacaccacttagacatacagagccgctcccacagaagccaccagacctgtgggggcaggggtggggcggttgttatgtggtaggtggggtcccccgtgcccacaccgttcctagggacccaagtcaccaccaaggctccaggtgagtagggaggaaggtggctcactcagcctgggactaggagcgggggctttgtggggagagctacaaagatggagacacacaaaacatcagagtggggaccagggacccagaggaggtgtgtgcctcgcttaaaatcacagtaccctgggccagacatagatgatgagggtgcagagagggtgtgtggcttgcagagggtcacacagcaccctgatggacaggaaaagagggctggggctgaaaggacttttacctttcccccagcttgacctctgaggcctgtcccagcaggtatctgtgggtccgccttggagagcaccacctctggaaatgggagggtccggagcagctgttccgggttacggacttcttcccccaccctggcttcaacaaggacctcagcgccaatgaccacaatgatgacatcatgctgatccgcctgcccaggcaggcacgtctgagtcctgctgtgcagcccctcaacctcagccagacctgtgtctccccaggcatgcagtgtctcatctcaggctggggggccgtgtccagccccaagggtatgacctggcccagaactctctctgaaacttgctccctcacccctctgtctctgccttttcatctctgtcttctccttttctctctcctctctctctctgtcagtctatctatctgccaatcgatatatttaaccaaatataagatgctagcatttttaagatgtgccattatttcatgaactgcgaagaagtggaagaaggaggaggaggagaagaaaaaaaggaggaggaggaaagatcccattagatcccattgattatataacaccattttctggaagacacattctaatttcagagtgtttgtttgtttgtttgtttgtttgtttttgagacagggtctcgctttgttgctcaggctggagtgcagcggtgtgatcacggctcattgcagctttgaactcctgggctcaagtgatcctctcgcctcaacctcccaagtagctgggattacagatatgcaccaccacatcccacaccggggtcatttttttattatttattattattattattattatctttttttttgtatttttagtagagacagaggtttcaccatattggccaggctggtctcaaattcctgacctggtgatctgcccgccttggactcccaaagtgctgggaaaacaggcatgagccactgcacccagccaaaattctagtcttttttaaatctagtcatatcttagatttaattcagataatgttagaatcctggagttttttgatccaggggaatctggaatgttagaatcttggattcataaaactctaaacgttgagcctctagattctagaatcatggatactagtgtgtcagaatctgagaattctagaatcttagattctgggcattctaatagtatcctggaatccacctgatgcaggaatcctctctccattgcctctgaaaagtgaccatccatactgttccaattttcttccctccatgaataaagcactgattctggtaaaagatgctgggtgggaatttcccatcatgcattgctccatgatgggacctcctttaacttaagccttatgctaaaaatttttattatttttagcaaagatgaggtcttgctatgttgtccaggctagtctcaaactcctggcctcccaaagtgctgagattacaagtgtgagccactgtacctggcccagagatgtttaaatgtgaaatgcgttcatcttagaatgggaataagaccatgtctctcagagtcacggatcactgacccattagccaaattgggtcagtggattggaaaaacagtctgaatttgttgctgccaatatctaaaacttggaaagttttatacaaaagccaggtttctggattcacctgaaaaagtttgaagaactcacattcccaaaatagcaagcattgggctgagtcaatggaggctgcccccttcagccaagataagttctctgattcactccaatggacccaaatggctcctgtctccctgcacagcccccgtccccgacttctgtttaccaattctgtttatcatatcccttgatgcatcggagcctgcacccatgtcttatatagatgcacatgtgtattatatatccatatccacatctatactgactacactgtatctggtatctctgtctatgtctctgtctccatcagtgaccatcttcctgcaaatctctttccttttatctcactgccttcattccaccccttgaggtctgggtctttttctatttcttttttttttttttttaagagactgagtcttgctcttgttgcccaggctggagtgcagtggtgtgatctcggctcactgcaacctccacctcctgggttttaagtgatcctcctgcctcagcctcccgagtagctgggactacaggtgtgcaacagcatgcccagctgattttttgtattttcagtagagacggagtttcaccatgttggccaggatggtctcaatctcttgaccttgtgatccgcccgcctcagcctcccaaagtgctagggagttatatatgcatctcctcttatctcttggctctctgcatgcatctttctgtttctcttccttcctttcttttttttttttttttttttttttttttttttttttttgagacggagtcttgctctgtctcccaggctggagtgcagtgaccagtctcggctcactgcaacctccacctcccaggttcaagtgattctcgtgcctcagcctcccgagtagctgggattacaggcgcctgccaccatgcctggctaatttttgtatatttagcagagatggggtttcaccatgttggctgggctggtctcaaactcctgacctcaagcgatccgccggcctcggcctccaaaacactgggattacaggcatgagccacggtgcccggccagcctctctctctacttggccctcttcctccttgtctccatttgtttctcttgtgtgctatgactgtctgtctgtcactgtctcttgtctctatctttgagagtcctaaatgtggctccattggtcctttggaaaagctgcagggaggactcagggcagtggggtgctgagtgtgttggagacagttgcagatccttgacagttctcttccctgacagcgctgtttccagtcacactgcagtgtgccaacatcagcatcctggagaacaaactctgtcactgggcataccctggacacatctcggacagcatgctctgtgcgggcctgtgggaggggggccgaggttcctgccaggtgagaccttactctggggaaaatgaggctgtcctgccaagttttctaggatttaggggagcagaggggtcggcccccagccttcctgggtcaaaatgagaaggagactgggatacctggttcctgggagaggacgggaccagggcctggactccttagtgtaaaagagaaaaggtctggaggtccagacttctggatctacaggaggagtgggctgggcgtccagagtctgagtcctcggggaggaggaggttaggtcctgcggggaggtgggccctctgagcttttactcctgggtctgaggaagaagaggctggagatggaggactctcggatgttggaggaggaaggggctggggcctttctgggagggaggaagtggcccgtgtaattgtcatgaacagagtggcctaacagttcctctgcccttctctcgcgtacagggtgactctgggggccccctggtttgcaatggaaccttggcaggcgtggtgtctgggggtgctgagccctgctccagaccccggcgccccgcagtctacaccagcgtatgccactaccttgactggatccaagaaatcatggagaactgagcccgcgcgccacgggggcaccttggaagaccaagagaggccgaagggcacggggtagggggttctcgtagggtcccagcctcaatggttcccgccctggacctccagctgccctgactcccctctggacactaagactccgcccctgaggctccgccccctcacgaggtcaagcaagacacagtcgcgccccctcggaacggagcagggacacgcccttcagagcccgtctctatgacgtcaccgacagccatcacctccttcttggaacagcacagcctgtggctccgccccaaggaaccacttacacaaaatagctccgcccctcggaactttgcccagtgggacttcccctcgggactccaccccttgtggccccgcctccttcaccagagatctcgcccctcgtgatgtcaggggcgcagtagctccgcccacgtggagctcgggcggtgtagagctcagccccttgtggccccgtcctgggcgtgtgctgggtttgaatcctggcggagacctggggggaaattgagggagggtctggatacctttagagccaatgcaacggatgatttttcagtaaacgcgggaaacctca

SEQ ID NO: 2 - protein, 234 aa, Homo sapiens
MKLGLLCALLSLLAGHGWADTRAIGAEECRPNSQPWQAGLFHLTRLFCGATLISDRWLLTAAHCRKPYLWVRLGEHHLWKWEGPEQLFRVTDFFPHPGFNKDLSANDHNDDIMLIRLPRQRCFQSHCSVPTSASWRTNSVTGHTLDTSRTACSVRACGRGAEVPARVTLGAPWFAMEPWQAWCLGVLSPAPDPGAPQSTPAYATTLTGSKKSWRTEPARHGGTLEDQERPKGTG

SEQ ID NO: 3 - protein, 250 aa, Homo sapiens
MKLGLLCALLSLLAGHGWADTRAIGAEECRPNSQPWQAGLFHLTRLFCGATLISDRWLLTAAHCRKPYLWVRLGEHHLWKWEGPEQLFRVTDFFPHPGFNKDLSANDHNDDIMLIRLPRQARLSPAVQPLNLSQTCVSPGMQCLISGWGAVSSPKALFPVTLQCANISILENKLCHWAYPGHISDSMLCAGLWEGGRGSCQGDSGGPLVCNGTLAGVVSGGAEPCSRPRRPAVYTSVCHYLDWIQEIMEN

SEQ ID NO: 4 - DNA, 20 bp, Homo sapiens
caagacccccctggatgtgg

SEQ ID NO: 5 - DNA, 21 bp, Homo sapiens
agttttcagagtccgtctcgg

SEQ ID NO: 6 - protein, 14 aa, Homo sapiens
CPHPGFNKDLSAND

(19) United States
(12) Patent Application Publication
(10) Pub. No.: US 2005/0176002 A1
(43) Pub. Date: Aug. 11, 2005

(54) Methods for detecting ovarian cancer

(75) Inventors: Eleftherios P Diamandis, Toronto (CA); George Yousef, Toronto (CA)

Correspondence Address: MERCHANT & GOULD PC, P.O. BOX 2903, MINNEAPOLIS, MN, 55402-0903, US

(73) Assignee: MOUNT SINAI HOSPITAL, TORONTO (CA)

(21) Appl. No.: 10/492,740

(22) PCT Filed: Oct. 16, 2002
(86) PCT No.: PCT/CA02/01555

Related U.S. Application Data
(60) Provisional application No. 60/330,099, filed on Oct. 16, 2001.

(51) Int. Cl. C12Q 1/68

(52) U.S. Cl. 435/6

(57) ABSTRACT

Methods for diagnosing and monitoring ovarian carcinoma in a subject are described comprising measuring KLK9 or hK9 in a sample from the subject. The invention also provides localization or imaging methods for ovarian cancer, and kits for carrying out the methods of the invention. The invention also contemplates therapeutic applications for ovarian cancer employing hK9 proteins, nucleic acid and molecules encoding the proteins, and/or binding agents for the proteins.

### FIELD OF THE INVENTION

The invention relates to methods for detecting ovarian cancer.

### BACKGROUND OF THE INVENTION

Ovarian cancer represents a great clinical challenge in gynecological oncology. Since most patients are asymptomatic until the disease has metastasized, two-thirds are diagnosed with advanced disease (1). In the United States, around 23,000 new cases of ovarian cancer and about 14,000 deaths from the disease were expected for the year 2000 (2), giving it the highest mortality rate of all gynecological malignancies.

Currently, the only tumor marker that has a well-defined and validated role in the management of ovarian cancer is CA125. Serum CA125 has been evaluated in the screening for ovarian cancer, differentiation between benign and malignant ovarian masses and prognosis (3-6). However, it does not yet have a clear place in diagnosis, prognosis, or in making treatment decisions (7, 8). In addition to ovarian cancer, high levels of CA125 were found in 1% of the normal population, 6% of patients with benign disease and 28% of patients with non-gynecological malignancies (9).

Many potential new serum markers have been evaluated, either alone or in combination with CA125, including CA15-3, CA19-9, OVX1, lysophosphatidic acid (LPA) and carcinoembryonic antigen (CEA) (7, 10, 11). These new markers do not have a well-defined contribution at present, and only the combination of CA125 with untrasonography yields the highest available sensitivity and specificity (8).

Kallireins are serine proteases with diverse physiological functions. Twelve new members of the human kallikrein gene family have recently been identified on chromosome 19q13.3-q13.4 (12-21). Several groups have shown that many human kallikrein genes are differentially expressed in various malignancies (reviewed in Ref. 22). PSA is the best marker for prostate cancer (23). hK2 (encoded by the KLK2 gene) is a useful marker for certain subgroups of patients (24-27). KLK10 (NES1) was found to be a tumor suppressor gene (28). The human stratum corneum chymotryptic enzyme (HSCCE) has been shown to be expressed at abnormally high levels in ovarian cancer (29), and KLK5 is a poor prognostic marker for ovarian cancer (30). Two new kallikrein proteins, hK6 and hK10 appear to be novel serological markers of ovarian carcinoma (31, 32).

KLX9 (formerly known as KLX-L3) is a newly identified member of the human kallikrein gene family (14, 33), expressed in many tissues including cerebellum, spinal cord, testis, prostate, ovary and skin. KLK9 was also found to be under steroid hormonal regulation in cancer cell lines (14). Interestingly, KLK8 (tumor-associated differentially expressed gene-14; TADG-14/neuropsin) and KLK10, the two genes flanking KLK9, were found to be differentially expressed in ovarian cancer (34-36). In addition, a very closely localized gene, KLK6, is also differentially expressed in primary ovarian tumors (19, 31).

### SUMMARY OF THE INVENTION

The present invention relates to novel biomarkers for ovarian cancer. The invention provides compositions and methods for the diagnosis and therapy of ovarian cancer.

KLK9, and proteins encoded by KLK9 have particular application in the detection of ovarian cancer. Thus, KLK9 constitutes a new biomarker for diagnosis and monitoring (i.e. monitoring progression or therapeutic treatment) of ovarian cancer.

In accordance with an aspect of the invention KLK9 is used for the diagnosis, monitoring, and prognosis of early stage (e.g. stage I or II), low grade (grade 1 or 2), optimally debulked ovarian cancer patients, and it may be used as a biomarker before surgery or after relapse. In another aspect of the invention, KLK9 is used for the diagnosis, monitoring, and prognosis of non-serous ovarian tumors.

KLK9 and fragments thereof, hK9, and agents that bind to hK9 may be used to detect ovarian cancer and they can be used in the diagnostic evaluation of ovarian cancer, and the identification of subjects with a predisposition to such disorders.

In an embodiment of the invention, a method is provided for detecting hK9 or KLK9 associated with ovarian cancer in a patient comprising:
(a) taking a sample from a patient;
(b) detecting or identifying in the sample hK9 or a KLK9 nucleic acid; and
(c) comparing the detected amount with an amount detected for a standard.

The term “detecting” includes assaying, imaging or otherwise establishing the presence or absence of the target hK9, KLK9, subunits thereof, or combinations of reagent bound targets, and the like, or assaying for, imaging, ascertaining, establishing, or otherwise determining one or more factual characteristics of ovarian cancer, metastasis, stage, or similar conditions. The term encompasses diagnostic, prognostic, and monitoring applications for hK9 and KLK9.

Methods for detecting KLK9 and hK9 can be used to monitor ovarian cancer by detecting hK9 and KLK9 nucleic acid molecules.

Methods of the invention may employ one or more polynucleotides, oligonucleotides, or nucleic acid molecules capable of hybridizing to a polynucleotide encoding hK9. Thus, methods for detecting KLK9 can be used to monitor ovarian cancer by detecting KLK9 nucleic acid molecules. In an aspect of the invention, KLK9 mRNA is detected.

Thus, the present invention relates to a method for diagnosing and monitoring ovarian cancer in a sample from a subject comprising isolating nucleic acids, preferably mRNA, from the sample; and detecting KLK9 nucleic acids in the sample. In an embodiment, the presence of increased levels of KLK9 nucleic acids in the sample compared to a standard or control is indicative of early disease stage, optimal debulking, and/or a positive prognosis i.e. longer progression-free and overall survival. The standard or control may be an amount associated with an advanced stage ovarian cancer.

The invention also provides methods for determining the presence or absence of ovarian cancer in a subject comprising detecting in the sample a level of polynucleotide that hybridizes to a nucleic acid molecule encoding hK9, comparing the level with a predetermined standard or cut-off value, and therefrom determining the presence or absence of ovarian cancer in the subject. In an embodiment a method is provided for determining the presence or absence of ovarian cancer in a subject comprising (a) contacting a sample obtained from the subject with an oligonucleotide that hybridizes to a nucleic acid molecule encoding hK9; and (b) detecting in the sample a level of polynucleotide that hybridizes to the nucleic acid molecule relative to a predetermined standard or cut-off value, and therefrom determining the presence or absence of ovarian cancer in the subject.

Within certain embodiments, the amount of polynucleotide that is mRNA is detected via amplification reactions such as polymerase chain reaction (PCR) using, for example, at least one oligonucleotide primer that hybridizes to a nucleic acid molecule that encodes hK9, or a complement of such nucleic acid molecule. (PCR techniques are well known in the art, see for example, U.S. Pat. Nos. 4,683,195 and 4,683,202 to Mullis). Within other embodiments, the amount of mRNA is detected using a hybridization technique, employing an oligonucleotide probe that hybridizes to hK9, or a complement of such nucleic acid molecule.

When using mRNA detection, the method may be carried out by combining isolated mRNA with reagents to convert to cDNA according to standard methods; treating the converted cDNA with amplification reaction reagents (such as cDNA PCR reaction reagents) in a container along with an appropriate mixture of nucleic acid primers; reacting the contents of the container to produce amplification products; and analyzing the amplification products to detect the presence of KLK9 marker in the sample. For mRNA the analyzing step may be accomplished using Northern Blot analysis to detect the presence of ovarian KLK9 marker. The analysis step may be further accomplished by quantitatively detecting the presence of KLK9 marker in the amplification product, and comparing the quantity of marker detected against a panel of expected values for known presence or absence in normal and malignant tissue derived using similar primers.

Therefore, the invention provides a method wherein mRNA is detected by (a) isolating mRNA from a sample and combining the mRNA with reagents to convert it to cDNA; (b) treating the converted cDNA with amplification reaction reagents and nucleic acid primers that hybridize to a nucleic acid molecule encoding hK9, to produce amplification products; (d) analyzing the amplification products to detect an amount of mRNA encoding hK9; and (e) comparing the amount of mRNA to an amount detected against a panel of expected values for normal and malignant tissue derived using similar nucleic acid primers.

The present invention further relates to a method for diagnosing and monitoring ovarian carcinoma in a subject comprising measuring hK9 in a sample from the subject hK9 may be measured using a reagent that detects or binds to hK9, preferably antibodies specifically reactive with hK9 or a part thereof.

In an aspect of the invention, a method for screening a subject for ovarian cancer is provided comprising (a) obtaining a biological sample from a subject; (b) detecting the amount of hK9 in said sample; and (c) comparing said amount of hK9 detected to a predetermined standard, where detection of a level of hK9 that differs from the standard indicates disease. In an embodiment the amount of hK9 detected is greater than that of a standard and indicates early disease stage, optimal debulking, and/or a positive prognosis i.e. longer progression-free and overall survival.

In a further embodiment, the invention provides methods for determining the presence or absence of ovarian cancer in a patient, comprising the steps of (a) contacting a biological sample obtained from a patient with a binding agent that specifically binds to hK9 protein; and (b) detecting in the sample an amount of protein that binds to the binding agent, relative to a predetermined standard or cut-off value, and therefrom determining the presence or absence of ovarian cancer in the patient.

In an embodiment, the invention relates to a method for diagnosing and monitoring ovarian cancer in a subject by quantitating hK9 in a biological sample from the subject comprising (a) reacting the biological sample with an antibody specific for hK9 which is directly or indirectly labelled with a detectable substance; and (b) detecting the detectable substance.

In another aspect the invention provides a method of using an antibody to detect expression of a hK9 protein in a sample, the method comprising: (a) combining an antibody specific for hK9 with a sample under conditions which allow the formation of antibody:protein complexes; and (b) detecting complex formation, wherein complex formation indicates expression of the protein in the sample. Expression may be compared with standards and is diagnostic of ovarian cancer.

Embodiments of the methods of the invention involve (a) reacting a biological sample from a subject with an antibody specific for hK9 which is directly or indirectly labelled with-an enzyme; (b) adding a substrate for the enzyme wherein the substrate is selected so that the substrate, or a reaction product of the enzyme and substrate forms fluorescent complexes; (c) quantitating hK9 in the sample by measuring fluorescence of the fluorescent complexes; and (d) comparing the quantitated levels to levels obtained for other samples from the subject patient, or control subjects. In an embodiment the quantitated levels are compared to levels quantitated for subjects with late stage ovarian cancer wherein an increase in hK9 levels compared with the control subjects is indicative of early stage disease, optimal debulling, and/or longer progression-free and overall survival.

A particular embodiment of the invention comprises the following steps
(a) incubating a biological sample with a first antibody specific for hK9 which is directly or indirectly labeled with a detectable substance, and a second antibody specific for hK9 which is immobilized;
(b) separating the first antibody from the second antibody to provide a first antibody phase and a second antibody phase;
(c) detecting the detectable substance in the first or second antibody phase thereby quantitating hK9 in the biological sample; and
(d) comparing the quantitated hK9 with levels for a predetermined standard.

The standard may correspond to levels quantitated for samples from control subjects with late stage disease or from other samples of the subject increased levels of hK9 as compared to the standard may be indicative of early stage ovarian cancer, optimal debulking, and/or longer progression-free and overall survival.

The invention also contemplates the methods described herein using multiple markers for ovarian cancer. Therefore, the invention contemplates a method for anaylzing a biological sample for the presence of hK9 and other markers that are specific indicators of ovarian cancer. Other markers include markers to kallikreins such as human stratum corneum chymotryptic enzyme (HSCCE), haptoglobin alpha, osteopontin, kallikrein 4 (hk4), kallikrein 5 (hK5), kallikrein 6 (hK6), kallikrein 8 (hK8), kallikrein 10, kallikrein 11, CA125, CA15-3, CA724, CA19-9, OVX1, lysophosphatidic acid (LPA), creatin-kinase BB, and carcinoembryonic antigen (CEA). Preferably the other markers are markers to kallikreins. In an aspect of the invention, the markers are one or more of hK4, hK5, hK6, hK8, HSCCE, and CA125, or nucleic acids encoding same. The methods described herein may be modified by including reagents to detect the markers, or nucleic acids for the markers.

The invention also provides a diagnostic composition comprising a hK9 protein or a nucleic acid molecule encoding the protein, or agents that bind to the protein or hybridize or amplify the nucleic acid molecule.

In an embodiment, the composition comprises a probe that specifically hybridizes to KLK9 or a fragment thereof. In another embodiment a composition is provided comprising a KLK9 specific primer pair capable of amplifying KLK9 using polymerase chain reaction methodologies. In a still further embodiment, the composition comprises an agent that binds to hK9 (e.g. antibody) or a fragment thereof. Probes, primers, and agents can be labeled with a detectable substance.

In accordance with an aspect of the invention an in vivo method is provided comprising administering to a subject an agent that has been constructed to target one or more kallikreins.

The invention therefore contemplates an in vivo method comprising administering to a mammal one or more agent that carries a label for imaging and binds to a kallikrein, preferably hK9, and then imaging the mammal.

According to a preferred aspect of the invention, an in vivo method for imaging ovarian cancer is provided comprising:
(a) injecting a patient with an agent that binds to kallikrein 9, the agent carrying a label for imaging the ovarian cancer;
(b) allowing the agent to incubate in vivo and bind to kallikrein 9 associated with the ovarian cancer; and
(c) detecting the presence of the label localized to the ovarian cancer.

In an embodiment of the invention the agent is an antibody which recognizes the kallikrein. In another embodiment of the invention the agent is a chemical entity which recognizes the kallikrein.

The agent carries a label to image the kallikreins. Examples of labels useful for imaging are radiolabels, fluorescent labels (e.g fluorescein and rhodamine), nuclear magnetic resonance active labels, positron emitting isotopes detectable by a positron emission tomography (“PET”) scanner, chemiluminescers such as luciferin, and enzymatic markers such as peroxidase or phosphatase. Short-range radiation emitters, such as isotopes detectable by short-range detector probes can also be employed.

The invention also contemplates the localization or imaging methods described herein using multiple markers for ovarian cancer. For example, a method for imaging ovarian cancer may further comprise injecting the patient with one or more of an agent that binds to human stratum corneum chymotryptic enzyme (HSCCE), haptoglobin alpha, osteopontin, kallikrein 4, kallikrein 5, kallikrein 6, kallikrein 8, kallikrein 10, kallikrein 11, CA125, CA72-4, CA15-3, CA19-9, OVX1, lysophosphatidic acid (LPA), creatin-kinase BB, or carcinoembryonic antigen (CEA).

The invention also relates to kits for carrying out the methods of the invention.

Still further the invention relates to therapeutic applications for ovarian cancer employing hK9 proteins, nucleic acid molecules encoding the proteins, and/or binding agents for the proteins.

In an aspect, the invention relates to a composition comprising one or more of a hK9 protein or part thereof, an antibody specific for hK9, or a nucleic acid molecule encoding hK9 or fragment thereof, and a pharmaceutically acceptable carrier, excipient or diluent. A method for treating or preventing ovarian cancer in a subject is also provided comprising administering to a patient in need thereof, a hK9 protein or part thereof, an antibody specific for hK9, or nucleic acid molecule encoding hK9 or fragment thereof, or a composition of the invention.

Another aspect of the invention is the use of a hK9 protein, peptides derived therefrom, or chemically produced (synthetic) peptides, or any combination of these molecules, for use in the preparation of vaccines to prevent ovarian cancer and/or to treat cancer.

The invention broadly contemplates vaccines for stimulating or enhancing in a subject to whom the vaccine is administered production of antibodies directed against a hK9 protein.

The invention also provides a method for stimulating or enhancing in a subject production of antibodies directed against hK9. The method comprises administering to the subject a vaccine of the invention in a dose effective for stimulating or enhancing production of the antibodies.

The invention further provides methods for treating, preventing, or delaying recurrence of ovarian cancer. The methods comprise administering to the subject a vaccine of the invention in a dose effective for treating, preventing, or delaying recurrence of cancer.

Other objects, features and advantages of the present invention will become apparent from the following detailed description. It should be understood, however, that the detailed description and the specific examples while indicating preferred embodiments of the invention are given by way of illustration only, since various changes and modifications within the spirit and scope of the invention will become apparent to those skilled in the art from this detailed description.

### DESCRIPTION OF THE DRAWINGS

The invention will now be described in relation to the drawings in which:

FIG. 1 shows the quantification of KLK9 gene expression by real-time PCR. (A): A logarithmic plot of fluorescence signal (Y-axis) versus cycle number (X-axis). Serial dilutions of a total RNA preparation from ovarian tissue were made and an arbitrary copy number was assigned to each sample, according to the dilution factor. Each sample was analyzed in duplicate. (B): A representative graph of the melting curve of the serial dilutions of the standard cDNA. The specific product melts at 92° C. This product was also run on agarose gel and sequenced to confirm the specificity of amplification.

FIG. 2 is a graph showing the determination of the optimal cut-off point value for KLK9 expression.

FIG. 3 shows Kaplan-Meier survival curves for patients with KLK9 positive and negative ovarian tumors. PFS, progression-free survival; OS, overall survival. n=number of samples.

FIG. 4 shows Kaplan-Meier survival curves for patients with KLK9 positive and negative tumors, stratified by tumor grade. PFS, progression-free survival; OS, overall survival. n=number of samples.

FIG. 5 shows Kaplan-Meier survival curves for patients with KLK9 positive and negative tumors, stratified by tumor stage. PFS, progression-free survival; OS, overall survival. n=number of samples.

FIG. 6 shows Kaplan-Meier survival curves for patients with KLK9 positive and negative tumors, stratified by the debulking success. PFS, progression-free survival; OS, overall survival. n=number of samples.

FIG. 7 shows the correlation between serum CA125 and tumor levels of KLK9. rₛ=Spearman correlation coefficient.

FIG. 8 shows the immunohistochemical localization of hK9 protein in a serous ovarian carcinoma. Moderate cytoplasmic positivity in tumor cells with no nuclear staining and negative stroma.

### DETAILED DESCRIPTION OF THE INVENTION

A variety of methods can be employed for the diagnostic and prognostic evaluation of ovarian cancer involving KLK9 and hK9, and the identification of subjects with a predisposition to such disorders. Such methods may, for example, utilize KLK9 nucleic acids, and fragments thereof, and binding agents (e.g. antibodies) directed against hK9, including peptide fragments. In particular, the nucleic acids and antibodies may be used, for example, for (1) the detection of the presence of KLK9 mutations, or the detection of either over- or under-expression of KLK9 mRNA relative to a non-disorder state or the qualitative or quantitative detection of alternatively spliced forms of KLK9 transcripts which may correlate with certain conditions or susceptibility toward such conditions; and (2) the detection of either an over- or an under-abundance of hK9 relative to a non-disorder state or the presence of a modified (e.g., less than full length) hK9 which correlates with a disorder state, or a progression toward a disorder state.

The methods described herein may be used to evaluate the probability of the presence of malignant or pre-malignant cells, for example, in a group of cells freshly removed from a host. Such methods can be used to detect tumors, quantitate their growth, and help in the diagnosis and prognosis of disease. The methods can be used to detect the presence of cancer metastasis, as well as confirm the absence or removal of all tumor tissue following surgery, cancer chemotherapy, and/or radiation therapy. They can further be used to monitor cancer chemotherapy and tumor reappearance.

The methods described herein can be adapted for diagnosing and monitoring ovarian carcinoma by detecting hK9 or KLK9 nucleic acid in biological samples from a subject. These applications require that the amount of hK9 or KLK9 nucleic acid quantitated in a sample from a subject being tested be compared to a predetermined standard or cut-off value. The standard may correspond to levels quantitated for another sample or an earlier sample from the subject, or levels quantitated for a control sample. Levels for control samples from healthy subjects or ovarian cancer subjects may be established by prospective and/or retrospective statistical studies. Healthy subjects who-have no clinically evident disease or abnormalities may be selected for statistical studies. Diagnosis may be made by a finding of statistically different levels of hK9 compared to a control sample or previous levels quantitated for the same subject.

The terms “sample”, “biological sample”, and the like mean a material known or suspected of expressing or containing KLK9 or hK9. The test sample can be used directly as obtained from the source or following a pretreatment to modify the character of the sample. The sample can be derived from any biological source, such as tissues, extracts, or cell cultures, including cells (e.g. tumor cells), cell lysates, and physiological fluids, such as, for example, whole blood, plasma, serum, saliva, ocular lens fluid, cerebral spinal fluid, sweat, urine, milk, ascites fluid, synovial fluid, peritoneal fluid and the like. The sample can be obtained from animals, preferably mammals, most preferably humans. The sample can be treated prior to use, such as preparing plasma from blood, diluting viscous fluids, and the like. Methods of treatment can involve filtration, distillation, extraction, concentration, inactivation of interfering components, the addition of reagents, and the like. Nucleic acids and proteins may be isolated from the samples and utilized in the methods of the invention.

The term “hK9” or “hK9 protein” refers to human kallikrein 9. The term includes all homologs, naturally occurring allelic variants, isoforms and precursors of human kallikrein 9 of GenBank Accession No AAD26427 and AF135026 (SEQ ID NO 2 and 3). In general for example, naturally occurring allelic variants of human kallikrein 9 will share significant homology (70-90%) to the sequences shown in GenBank Accession No AAD26427 and AF135026 (SEQ ID NO 2 and 3). Allelic variants may contain conservative amino acid substitutions from the hK9 sequence herein described or will contain a substitution of an amino acid from a corresponding position in a hK9 homologue such as, for example, the murine hK9 homologue.

“KLK9” or “KLK9 nucleic acid molecule(s)” refers to a polynucleotide encoding a hK9, preferably having the sequence of GenBank Accession No. AF135026 (SEQ ID NO 1), or a variant thereof. Variants preferably exhibit at least about 70%, preferably at least about 90% identity to a polynucleotide sequence that encodes a native kallikrein 9 or a portion thereof. Certain variants are substantially homologous to a native KLK9 gene, or a portion or complement thereof. For example, such variants are capable of hybridizing under moderate stringency conditions to a naturally occurring DNA sequence encoding a hK9 protein (or a complementary sequence). Suitable moderate stringency conditions include prewashing with a solution of 5×SSC, 0.5% SDS, 1.0 mM EDTA (pH 8.0); hybridizing at 50° C.-56° C. for 20 minutes with each of 2×, 0.5×, and 0.2×SSC containing 0.1% SDS.

The term “KLK9” or “KLK9 nucleic acid molecule(s)” is intended to include DNA and RNA (e.g. mRNA) and can be either double stranded or single stranded. A polynucleotide may, but need not, include additional coding or non-coding sequences, or it may, but need not, be linked to other molecules and/or carrier or support materials. The nucleic acid molecules for use in the methods of the invention may be of any length suitable for a particular method. In certain applications the term refers to antisense nucleic acid molecules (e.g. an mRNA or DNA strand in the reverse orientation to a sense KLK9 molecule).

The term “subject” or “patient” refers to a warm-blooded animal such as a mammal that is afflicted with, suspected of being afflicted with, or being screened for ovarian cancer or condition as described herein. Preferably, “subject” refers to a human.

Nucleic Acid Methods

As noted herein an ovarian cancer may be detected based on the level of KLK9 in a sample. Techniques for detecting nucleic acid molecules such as polymerase chain reaction (PCR) and hybridization assays are well known in the art.

A probe may be used in hybridization techniques to detect KLK9 nucleic acids. The technique generally involves contacting and incubating nucleic acids obtained from a sample from a patient or other cellular source with a probe under conditions favorable for the specific annealing of the probes to complementary sequences in the nucleic acids. After incubation, the non-annealed nucleic acids are removed, and the presence of nucleic acids that have hybridized to the probe if any are detected.

Nucleotide probes for use in the detection of KLK9 nucleic acid sequences in samples may be constructed using conventional methods known in the art Suitable probes are based on nucleic acid sequences encoding at least 5 sequential amino acids from regions of KLK9 nucleic acid, preferably they comprise 15 to 30 nucleotides. A nucleotide probe may be labeled with a detectable substance such as a radioactive label which provides for an adequate signal and has sufficient half-life such as ³²P, ³H, ¹⁴C or the like. Other detectable substances which may be used include antigens that are recognized by a specific labeled antibody, fluorescent compounds, enzymes, antibodies specific for a labeled antigen, and luminescent compounds. An appropriate label may be selected having regard to the rate of hybridization and binding of the probe to the nucleotide to be detected and the amount of nucleotide available for hybridization. Labeled probes may be hybridized to nucleic acids on solid supports such as nitrocellulose filters or nylon membranes as generally described in Sambrook et al, 1989, Molecular Cloning, A Laboratory Manual (2nd ed.). The nucleic acid probes may be used to detect KLK9 nucleic acids, preferably in human cells. The nucleotide probes may also be useful in the diagnosis of ovarian cancer involving KLK9, in monitoring the progression of such disorder, or monitoring a therapeutic treatment.

The detection of KLK9 nucleic acid may involve the amplification of specific gene sequences using an amplification method such as PCR, followed by the analysis of the amplified molecules using techniques known to those skilled in the art. Suitable primers can be routinely designed by one of skill in the art.

By way of example, at least two oligonucleotide primers may be employed in a PCR based assay to amplify a portion of a nucleic acid molecule encoding hK9 derived from a sample, wherein at least one of the oligonucleotide primers is specific for (i.e. hybridizes to) a polynucleotide encoding hK9. The amplified cDNA is then separated and detected using techniques well known in the art, such as gel electrophoresis.

In order to maximize hybridization under assay conditions, primers and probes employed in the methods of the invention generally have at least about 60%, preferably at least about 75% and more preferably at least about 90% identity to a portion of a polynucleotide encoding hK9; that is, they are at least 10 nucleotides, and preferably at least 20 nucleotides in length. In an embodiment the primers and probes are at least about 10-40 nucleotides in length.

Hybridization and amplification techniques described herein may be used to assay qualitative and quantitative aspects of KLK9 nucleic acid expression. For example, RNA may be isolated from a cell type or tissue known to express KLK9 and tested utilizing the hybridization (e.g. standard Northern analyses) or PCR techniques referred to herein.

The primers and probes may be used in the above described methods in situ i.e directly on tissue sections (fixed and/or frozen) of patient tissue obtained from biopsies or resections.

In an aspect of the invention, a method is provided employing reverse transcriptase-polymerase chain reaction (RT-PCR), in which PCR is applied in combination with reverse transcription. Generally, RNA is extracted from a sample tissue using standard techniques (for example, guanidine isothiocyanate extraction as described by Chomcynski and Sacchi, Anal. Biochem. 162:156-159, 1987) and is reverse transcribed to produce cDNA. The cDNA is used as a template for a polymerase chain reaction. The cDNA is hybridized to a set of primers, at least one of which is specifically designed against a hK9 sequence. Once the primer and template have annealed a DNA polymerase is employed to extend from the primer, to synthesize a copy of the template. The DNA strands are denatured, and the procedure is repeated many times until sufficient DNA is generated to allow visualization by ethidium bromide staining and agarose gel electrophoresis.

Amplification may be performed on samples obtained from a subject with suspected ovarian cancer and an individual who is not afflicted with ovarian cancer or has advanced stage disease. The reaction may be performed on several dilutions of cDNA spanning at least two orders of magnitude. A statistically significant difference in expression in several dilutions of the subject sample as compared to the same dilutions of the non-cancerous sample or late-stage cancer sample may be considered positive for the presence of ovarian cancer.

Oligonucleotides or longer fragments derived from a KLK9 nucleic acid may be used as targets in a microarray. The microarray can be used to simultaneously monitor the expression levels of large numbers of genes and to identify genetic variants, mutations, and polymorphisms. The information from the microarray may be used to determine gene function, to understand the genetic basis of a disorder, to diagnose a disorder, and to develop and monitor the activities of therapeutic agents.

The preparation, use, and analysis of microarrays are well known to a person skilled in the art. (See, for example, Brennan, T. M. et al. (1995) U.S. Pat. No. 5,474,796; Schena, et al. (1996) Proc. Natl. Acad. Sci. 93:10614-10619; Baldeschweiler et al. (1995), PCT Application WO95/251116; Shalon, D. et al. (1995) PCT application WO95/35505; Heller, R. A. et al. (1997) Proc. Natl. Acad. Sci. 94:2150-2155; and Heller, M. J. et al. (1997) U.S. Pat. No. 5,605,662.)

Protein Methods

Binding agents may be used for a variety of diagnostic and assay applications. There are a variety of assay formats known to the skilled artisan for using a binding agent to detect a target molecule in a sample. (For example, see Harlow and Lane, Antibodies: A Laboratory Manual, Cold Spring Harbor Laboratory, 1988). In general, the presence or absence of a cancer in a subject may be determine by (a) contacting a sample from the subject with a binding agent; (b) detecting in the sample a level of polypeptide that binds to the binding agent; and (c) comparing the level of polypeptide with a predetermined standard or cut-off value.

“Binding agent” refers to a substance such as a polypeptide or antibody that specifically binds to a hK9 protein. A substance “specifically binds” to a hK9 protein if it reacts at a detectable level with hK9, and does not react detectably with peptide containing an unrelated sequence or a sequence of a different kallikrein. Binding properties may be assessed using an ELISA, which may be readily performed by those skilled in the art (see for example, Newton et al, Develop. Dynamics 197: 1-13, 1993).

A binding agent may be a ribosome, with or without a peptide component, an RNA molecule, or a polypeptide. A binding agent may be a polypeptide that comprises an hK9 sequence, a peptide variant thereof, or a non-peptide mimetic of such a sequence. By way of example a hK9 sequence may be a peptide portion of a hK9 that is capable of modulating a function mediated by hK9.

In certain other preferred embodiments, the binding agent is an antibody. Antibodies specifically reactive with a hK9 protein, or derivatives, such as enzyme conjugates or labeled derivatives, may be used to detect hK9 protein in various samples (e.g. biological materials). They may be used as diagnostic or prognostic reagents and they may be used to detect abnormalities in the level of hK9 expression, or abnormalities in the structure, and/or temporal, tissue, cellular, or subcellular location of hK9. Antibodies may also be used to screen potentially therapeutic compounds in vitro to determine their effects on disorders (e.g. ovarian cancer) involving a hK9 protein, and other conditions. In vitro immunoassays may also be used to assess or monitor the efficacy of particular therapies.

In an aspect, the invention provides a diagnostic method for monitoring or diagnosing ovarian carcinoma in a subject by quantitating hK9 in a biological sample from the subject comprising reacting the sample with an antibody specific for hK9 which is directly or indirectly labeled with a detectable substance, and detecting the detectable substance.

In an embodiment, the invention contemplates a method for monitoring the progression of ovarian cancer in an individual, comprising:
(a) contacting an amount of an antibody which binds to a hK9 protein, with a sample from the individual so as to form a binary complex comprising the antibody and hK9 protein in the sample;
(b) determining or detecting the presence or amount of complex formation in the sample;
(c) repeating steps (a) and (b) at a point later in time; and
(d) comparing the result of step (b) with the result of step (c), wherein a difference in the amount of complex formation is indicative of the stage and/or progression of the ovarian cancer in said individual.

The amount of complexes may also be compared to a value representative of the amount of the complexes from an individual not at risk of, or afflicted with, ovarian cancer at different stages.

Antibodies may be used in any known immunoassays that rely on the binding interaction between an antigenic determinant of a hK9 protein and the antibodies. Examples of such assays are radioimmunoassays, enzyme immunoassays (e.g. ELISA), immunofluorescence, immunoprecipitation, latex agglutination, hemagglutination, and histochemical tests. These terms are well understood by those skilled in the art. A person skilled in the art will know, or can readily discern, other immunoassay formats without undue experimentation.

The antibodies may be used in immunohistochemical analyses, for example, at the cellular and sub-subcellular level, to detect a hK9 protein, to localize it to particular ovarian tumor cells and tissues, and to specific subcellular locations, and to quantitate the level of expression.

An antibody for use in the present invention includes a monoclonal or polyclonal antibody, an immunologically active fragment (e.g. a Fab or (Fab)₂ fragment), an antibody heavy chain, humanized antibody, an antibody light chain, a genetically engineered single chain Fᵥ molecule (Ladner et al, U.S. Pat. No. 4,946,778), a chimeric antibody, for example, an antibody which contains the binding specificity of a murine antibody, but in which the remaining portions are of human origin, or derivatives, such as enzyme conjugates or labeled derivatives.

Antibodies including monoclonal and polyclonal antibodies, fragments and chimeras, may be prepared using methods known to those skilled in the art. Isolated native hK9 or recombinant hK9 may be utilized to prepare antibodies. See, for example, Kohler et al. (1975) Nature 256:495-497; Kozbor et al. (1985) J. Immunol Methods 81:31-42; Cote et al. (1983) Proc Natl Acad Sci 80:2026-2030; and Cole et al. (1984) Mol Cell Biol 62:109-120 for the preparation of monoclonal antibodies; Huse et al. (1989) Science 246:1275-1281 for the preparation of monoclonal Fab fragments; and, Pound (1998) Immunochemical Protocols, Humana Press, Totowa, N.J. for the preparation of phagemid or B-lymphocyte immunoglobulin libraries to identify antibodies. The antibodies specific for hK9 used in the methods of the invention may also be obtained from scientific or commercial sources.

In an embodiment, antibodies are reactive against hK9 if they bind with a Kₐ of greater than or equal to 10⁻⁷ M. In a sandwich immunoassay of the invention mouse polyclonal antibodies and rabbit polyclonal antibodies may be utilized.

An antibody that binds to hK9 may be labelled with a detectable substance and localised in biological samples based upon the presence of the detectable substance. Examples of detectable substances include, but are not limited to, the following: radioisotopes (e.g., ³H, ¹⁴C, ³⁵S, ¹²⁵I, ¹³¹I), fluorescent labels (e.g., FITC, rhodamine, lanthanide phosphors); luminescent labels such as luminol; enzymatic labels (e.g., horseradish peroxidase, beta-galactosidase, luciferase, alkaline phosphatase, acetylcholinesterase); biotinyl groups (which can be detected by marked avidin e.g., streptavidin containing a fluorescent marker or enzymatic activity that can be detected by optical or calorimetric methods); and, predetermined polypeptide epitopes recognized by a secondary reporter (e.g., leucine zipper pair sequences, binding sites for secondary antibodies, metal binding domains, epitope tags). In some aspects, labels are attached via spacer arms of various lengths to reduce potential steric hindrance. Antibodies may also be coupled to electron dense substances, such as ferritin or colloidal gold, which are readily visualised by electron microscopy.

Indirect methods may also be employed in which the primary antigen-antibody reaction is amplified by the introduction of a second antibody, having specificity for the antibody reactive against hK9. By way of example, if the antibody having specificity against hK9 is a rabbit IgG antibody, the second antibody may be goat anti-rabbit gamma-globulin labelled with a detectable substance as described herein.

Methods for conjugating or labelling the antibodies discussed above may be readily accomplished by one of ordinary skill in the art (See for example Inman, Methods In Enzymology, Vol. 34, Affinity Techniques, Enzyme Purification: Part B, Jakoby and Wichek (eds.), Academic Press, New York, p. 30, 1974; and Wilchek and Bayer, “The Avidin-Biotin Complex in Bioanalytical Applications,” Anal. Biochem. 171:1-32, 1988 re methods for conjugating or labelling the antibodies with enzyme or ligand binding partner).

Cytochemical techniques known in the art for localizing antigens using light and electron microscopy may be used to detect a hK9 protein. Generally, an antibody may be labeled with a detectable substance and hK9 protein may be localised in tissues and cells based upon the presence of the detectable substance.

In the context of the methods of the invention, the sample or a binding agent (e.g. antibody) for hK9 may be immobilized on a carrier or support Examples of suitable carriers or supports are agarose, cellulose, nitrocellulose, dextran, Sephadex, Sepharose, liposomes, carboxymethyl cellulose, polyacrylamides, polystyrene, gabbros, filter paper, magnetite, ion-exchange resin, plastic film, plastic tube, glass, polyamine-methyl vinyl-ether-maleic acid copolymer, amino acid copolymer, ethylene-maleic acid copolymer, nylon, silk, etc. The support material may have any possible configuration including spherical (e.g. bead), cylindrical (e.g. inside surface of a test tube or well, or the external surface of a rod), or flat (e.g. sheet, test strip). Thus, the carrier may be in the shape of, for example, a tube, test plate, well, beads, disc, sphere, etc. The immobilized material may be prepared by reacting the material with a suitable insoluble carrier using known chemical or physical methods, for example, cyanogen bromide coupling. A binding agent (e.g. antibody) may be indirectly immobilized using a second antibody specific for the antibody. For example, mouse anti-hK9 antibody may be immobilized using sheep anti-mouse IgG Fc fragment specific antibody coated on the carrier or support.

Where a radioactive label is used as a detectable substance, a hK9 protein may be localized by radioautography. The results of radioautography may be quantitated by determining the density of particles in the radioautographs by various optical methods, or by counting the grains.

Time-resolved fluorometry may be used to detect a signal. For example, the method described in Christopoulos T K and Diamandis E P Anal Chem 1992:64:342-346 may be used with a conventional time-resolved fluorometer.

Therefore, in accordance with an embodiment of the invention, a method is provided wherein a hK9 antibody is labelled with an enzyme, a substrate for the enzyme is added wherein the substrate is selected so that the substrate, or a reaction product of the enzyme and substrate, forms fluorescent complexes with a lanthanide metal. A lanthanide metal is added and hK9 is quantitated in the sample by measuring fluorescence of the fluorescent complexes. The antibodies specific for hK9 may be directly or indirectly labelled with an enzyme. Enzymes are selected based on the ability of a substrate of the enzyme, or a reaction product of the enzyme and substrate, to complex with lanthanide metals such as europium and terbium.

Examples of enzymes and substrates for enzymes that provide such fluorescent complexes are described in U.S. Pat. No. 5,312,922 to Diamandis. In an aspect of the invention the enzyme is alkaline phosphatase or β-galactosidase. In an embodiment, the enzyme is alkaline phosphatase. By way of example, when the antibody is directly or indirectly labelled with alkaline phosphatase the substrate employed in the method may be 4-methylumbelliferyl phosphate, 5-fluorosalicyl phosphate, or diflunisal phosphate. The fluorescence intensity of the complexes is typically measured using a time-resolved fluorometer e.g. a CyberFluor 615 Imunoanalyzer (Nordion International, Kanata, Ontario).

The hK9 antibodies may also be indirectly labelled with an enzyme. For example, the antibodies may be conjugated to one partner of a ligand binding pair, and the enzyme may be coupled to the other partner of the ligand binding pair. Representative examples include avidin-biotin, and riboflavin-riboflavin binding protein. Preferably the antibodies are biotinylated, and the enzyme is coupled to streptavidin. In another embodiment, an antibody specific for the anti-hK9 antibody is labeled with an enzyme.

In accordance with an embodiment, the present invention provides means for determining hK9 in a serum sample by measuring hK9 by immunoassay. It will be evident to a skilled artisan that a variety of immunoassay methods can be used to measure hK9 in serum. In general, a hK9 immunoassay method may be competitive or noncompetitive. Competitive methods typically employ an immobilized or immobilizable antibody to hK9 (anti-hK9) and a labeled form of hK9. Sample hK9 and labeled hK9 compete for binding to anti-hK9. After separation of the resulting labeled hK9 that has become bound to anti-hK9 (bound fraction) from that which has remained unbound (unbound fraction), the amount of the label in either bound or unbound fraction is measured and may be correlated with the amount of hK9 in the test sample in any conventional manner, e.g., by comparison to a standard curve.

Preferably a non-competitive method is used for the determination of hK9, with the most common method being the “sandwich” method. In this assay, two anti-hK9 antibodies are employed. One of the anti-hK9 antibodies is directly or indirectly labeled (sometimes referred to as the “detection antibody”) and the other is immobilized or immobilizable (sometimes referred to as the “capture antibody”). The capture and detection antibodies can be contacted simultaneously or sequentially with the test sample. Sequential methods can be accomplished by incubating the capture antibody with the sample, and adding the detection antibody at a predetermined time thereafter (sometimes referred to as the “forward” method); or the detection antibody can be incubated with the sample first and then the capture antibody added (sometimes referred to as the “reverse” method). After the necessary incubation(s) have occurred, to complete the assay, the capture antibody is separated from the liquid test mixture, and the label is measured in at least a portion of the separated capture antibody phase or the remainder of the liquid test mixture. Generally it is measured in the capture antibody phase since it comprises hK9 bound by (“sandwiched” between) the capture and detection antibodies.

In a typical two-site immunometric assay for hK9, one or both of the capture and detection antibodies are polyclonal antibodies, or one or both of the capture and detection antibodies are monoclonal antibodies (i.e. polyclonal/polyclonal, monoclonal/monoclonal, or monoclonal/polyclonal). The label used in the detection antibody can be selected from any of those known conventionally in the art. The label may be an enzyme or a chemiluminescent moiety, but it can also be a radioactive isotope, a fluorophor, a detectable ligand (e.g., detectable by a secondary binding by a labeled binding partner for the ligand), and the like. Preferably the antibody is labelled with an enzyme which is detected by adding a substrate that is selected so that a reaction product of the enzyme and substrate forms fluorescent complexes. The capture antibody is selected so that it provides a means for being separated from the remainder of the test mixture. Accordingly, the capture antibody can be introduced to the assay in an already immobilized or insoluble form, or can be in a immobilizable form, that is, a form which enables immobilization to be accomplished subsequent to introduction of the capture antibody to the assay. An immobilized capture antibody may comprise an antibody covalently or noncovalently attached to a solid phase such as a magnetic particle, a latex particle, a microtiter plate well, a bead, a cuvette, or other reaction vessel. An example of an immobilizable capture antibody is antibody which has been chemically modified with a ligand moiety, e.g., a hapten, biotin, or the like, and which can be subsequently immobilized by contact with an immobilized form of a binding partner for the ligand, e.g., an antibody, avidin, or the like. In an embodiment, the capture antibody may be immobilized using a species specific antibody for the capture antibody that is bound to the solid phase.

A particular sandwich immunoassay method of the invention employs two antibodies reactive against hK9, a second antibody having specificity against an antibody reactive against hK9 labelled with an enzymatic label, and a fluorogenic substrate for the enzyme. In an embodiment, the enzyme is alkaline phosphatase (ALP) and the substrate is 5-fluorosalicyl phosphate. ALP cleaves phosphate out of the fluorogenic substrate, 5-fluorosalicyl phosphate, to produce 5-fluorosalicylic acid (FSA). 5-Fluorosalicylic acid can then form a highly fluorescent ternary complex of the form FSA-Tb(3+)-EDTA, which can be quantified by measuring the Tb3+ fluorescence in a time-resolved mode. Fluorescence intensity is measured using a time-resolved fluorometer as described herein.

The above-described immunoassay methods and formats are intended to be exemplary and are not limiting since, in general, it will be understood that any immunoassay method or format can be used in the present invention.

Imaging Methods

Binding agents, in particular antibodies, that bind hK9 may be used in imaging methodologies in the management of ovarian cancer. The invention provides a method for imaging tumors associated with one or more kallikreins, preferably kallilcreins associated with ovarian cancer, most preferably hK9.

The invention also contemplates imaging methods described herein using multiple markers for ovarian cancer. For example, a method for imaging ovarian cancer may further comprise injecting the patient with one or more of an agent that binds to human stratum corneum chymotryptic enzyme (HSCCE), haptoglobin alpha, osteopontin, kallikrein 4, kalikrein 5, kallikrein 6, kallikrein 8, kallikrein 10, kallikrein 11, CA125, CA15-3, CA19-9, CA72-4, OVX1, lysophosphatidic acid (LPA), creatin-kinase BB, or carcinoembryonic antigen (CEA). Preferably each agent is labeled so that it can be distinguished during the imaging.

In an embodiment the method is an in vivo method and a subject or patient is administered one or more agents that carry an imaging label and that are capable of targeting or binding to a kallikrein. The agent is allowed to incubate in vivo and bind to the kallikrein(s) associated with a tumor, preferably ovarian tumors. The presence of the label is localized to the ovarian cancer, and the localized label is detected using imaging devices known to those skilled in the art.

The binding agent may be an antibody or chemical entity that recognizes the kallikrein(s). In an aspect of the invention the agent is a polyclonal antibody or monoclonal antibody, or fragments thereof, or constructs thereof including but not limited to, single chain antibodies, bifunctional antibodies, molecular recognition units, and peptides or entities that mimic peptides. The antibodies specific for the kallikreins used in the methods of the invention may be obtained from scientific or commercial sources, or isolated native kallikrein or recombinant kallikrein may be utilized to prepare antibodies etc as described herein.

An agent may be a peptide that mimics the epitope for an antibody specific for a kallikrein and binds to the kallikrein. The peptide may be produced on a commercial synthesizer using conventional solid phase chemistry. By way of example, a peptide may be prepared that includes either tyrosine lysine, or phenylalanine to which N₂S₂ chelate is complexed (See U.S. Pat. No. 4,897,255). The anti-kallikrein peptide conjugate is then combined with a radiolabel (e.g. sodium ^{99m}Tc pertechnetate or sodium ¹⁸⁸Re perrhenate) and it may be used to locate a kallilkrein producing tumor.

The agent carries a label to image the kallikreins. The agent may be labelled for use in radionuclide imaging. In particular, the agent may be directly or indirectly labelled with a radioisotope. Examples of radioisotopes that may be used in the present invention are the following: ²⁷⁷Ac, ²¹¹At, ¹²⁸Ba, ¹³¹Ba, ⁷Be, ²⁰⁴Bi, ²⁰⁵Bi, ²⁰⁶Bi, ⁷⁶Br, ⁷⁷Br, ⁸²Br, ¹⁰⁹Cd, ⁴⁷Ca, ¹¹C, ¹⁴C, ³⁶Cl, ⁴⁸Cr, ⁵¹Cr, ⁶²Cu, ⁶⁴Cu, ⁶⁷Cu, ¹⁶⁵Dy, ¹⁵⁵Eu, ¹⁸F, ¹⁵³Gd, ⁶⁶Ga, ⁶⁷Ga, ⁶⁸Ga, ⁷²Ga, ¹⁹⁸Au, ³H, ¹⁶⁶Ho, ¹¹¹In, ^{113m}In, ^{115m}In, ¹²³I, ¹²⁵I, ¹³¹I, ¹⁸⁹Ir, ^{191m}Ir, ¹⁹²Ir, ¹⁹⁴Ir, ⁵²Fe, ⁵⁵Fe, ⁵⁹Fe, ¹⁷⁷Lu, ¹⁵O, ^{119m-191}Os, ¹⁰⁹Pd, ³²P, ³³P, ⁴²K, ²²⁶Ra, ¹⁸⁶Re, ¹⁸⁸Re, ^{82m}Rb, ¹⁵³Sm, ⁴⁶Sc, ⁴⁷Sc, ⁷²Se, ⁷⁵Se, ¹⁰⁵Ag, ²²Na, ²⁴Na, ⁸⁹Sr, ³⁵S, ³⁸S, ¹⁷⁷Ta, ⁹⁶Tc, ^{99m}Tc, ²⁰¹Tl, ²⁰²Tl, ¹¹³Sn, ^{117m}Sn, ¹²¹Sn, ¹⁶⁶Yb, ¹⁶⁹Yb, ¹⁷⁵Yb, ⁸⁸Y, ⁹⁰Y, ⁶³Zn and ⁶⁵Zn. Preferably the radioisotope is ¹³¹I, ¹²⁵I, ¹²³I, ¹¹¹I, ^{99m}Tc, ⁹⁰Y, ¹⁸⁶Re, ¹⁸⁸Re, ³²P, ¹⁵³Sm, ⁶⁷Ga, ²⁰¹Tl ⁷⁷Br, or ¹⁸F, and is imaged with a photoscanning device.

Procedures for labeling biological agents with the radioactive isotopes are generally known in the art. U.S. Pat. No. 4,302,438 describes tritium labeling procedures. Procedures for iodinating, tritium labeling, and ³⁵S labeling especially adapted for murine monoclonal antibodies are described by Goding, J. W. (supra, pp 124-126) and the references cited therein. Other procedures for iodinating biological agents, such as antibodies, binding portions thereof, probes, or ligands, are described in the scientific literature (see Hunter and Greenwood, Nature 144:945 (1962), David et al., Biochemistry 13:1014-1021 (1974), and U.S. Pat. Nos. 3,867,517 and 4,376,110). Iodinating procedures for agents are described by Greenwood, F. et al., Biochem. J. 89:114-123 (1963); Marchalonis, J., Biochem. J. 113:299-305 (1969); and Morrison, M. et al., Immunochemistry, 289-297 (1971). ^{99m}Tc-labeling procedures are described by Rhodes, B. et al. in Burchiel, S. et al. (eds.), Tumor Imaging: The Radioimmunochemical Detection of Cancer, New York: Masson 111-123 (1982) and the references cited therein. Labelling of antibodies or fragments with technetium-99m are also described for example in U.S. Pat. No. 5,317,091, U.S. Pat. No. 4,478,815, U.S. Pat. No. 4,478,818, U.S. Pat. No. 4,472,371, U.S. Pat. No. Re 32,417, and U.S. Pat. No. 4,311,688. Procedures suitable for ¹¹¹In-labeling biological agents are described by Hnatowich, D. J. et al., J. Immul. Methods, 65:147-157 (1983), Hnatowich, D. et al., J. Applied Radiation, 35:554-557 (1984), and Buckley, R. G. et al., F.E.B.S. 166:202-204 (1984).

An agent may also be labeled with a paramagnetic isotope for purposes of an in vivo method of the invention. Examples of elements that are useful in magnetic resonance imaging include gadolinium, terbium, tin, iron, or isotopes thereof. (See, for example, Schaefer et al., (1989) JACC 14, 472-480; Shreve et al., (1986) Magn. Reson. Med. 3, 336-340; Wolf, G L., (1984) Physiol. Chem. Phys. Med. NMR 16, 93-95; Wesbey et al., (1984) Physiol. Chem. Phys. Med. NMR 16, 145-155; Runge et al., (1984) Invest. Radiol. 19, 408-415 for discussions on in vivo nuclear magnetic resonance imaging.)

In the case of a radiolabeled agent, the agent may be administered to the patient, it is localized to the tumor having a kallikrein with which the agent binds, and is detected or “imaged” in vivo using known techniques such as radionuclear scanning using e.g., a gamma camera or emission tomography. [See for example A. R. Bradwell et al., “Developments in Antibody Imaging”, Monoclonal Antibodies for Cancer Detection and Therapy, R. W. Baldwin et al., (eds.), pp. 65-85 (Academic Press 1985)]. A positron emission transaxial tomography scanner, such as designated Pet VI located at Brookhaven National Laboratory, can also be used where the radiolabel emits positrons (e.g., ¹¹C, ¹⁸F, ¹⁵O, and ¹³N).

Whole body imaging techniques using radioisotope labeled agents can be used for locating both primary tumors and tumors which have metastasized. Antibodies specific for kallikreins, or fragments thereof having the same epitope specificity, are bound to a suitable radioisotope, or a combination thereof, and administered parenterally. For ovarian cancer, administration preferably is intravenous. The bio-distribution of the label can be monitored by scintigraphy, and accumulations of the label are related to the presence of ovarian cancer cells. Whole body imaging techniques are described in U.S. Pat. Nos. 4,036,945 and 4,311,688. Other examples of agents useful for diagnosis and therapeutic use which can be coupled to antibodies and antibody fragments include metallothionein and fragments (see, U.S. Pat. No. 4,732,864). These agents are useful in diagnosis staging and visualization of cancer, in particular ovarian cancer, so that surgical and/or radiation treatment protocols can be used more efficiently.

The agent may carry a bioluminescent or chemiluminescent label. Such labels include polypeptides known to be fluorescent, bioluminescent or chemiluminescent, or, that act as enzymes on a specific substrate (reagent), or can generate a fluorescent, bioluminescent or chemiluminescent molecule. Examples of bioluminescent or chemiluminescent labels include luciferases, aequorin, obelin, mnemiopsin, berovin, a phenanthridinium ester, and variations thereof and combinations thereof. A substrate for the bioluminescent or chemiluminescent polypeptide may also be utilized in a method of the invention. For example, the chemiluminescent polypeptide can be luciferase and the reagent luciferin. A substrate for a bioluminescent or chemiluminescent label can be administered before, at the same time (e.g., in the same formulation), or after administration of the agent.

An agent may comprise a paramagnetic compound, such as a polypeptide chelated to a metal, e.g., a metalloporphyrin. The paramagnetic compound may also comprise a monocrystalline nanoparticle, e.g., a nanoparticle comprising a lanthanide (e.g., Gd) or iron oxide; or, a metal ion comprising a lanthanide. “Lanthanides” refers to elements of atomic numbers 58 to 70, a transition metal of atomic numbers 21 to 29, 42 or 44, a Gd(III), a Mn(II), or an element comprising an Fe element. Paramagnetic compounds can also comprise a neodymium iron oxide (NdFeO.sub.3) or a dysprosium iron oxide (DyFeO.sub.3). Examples of elements that are useful in magnetic resonance imaging include gadolinium, terbium, tin, iron, or isotopes thereof. (See, for example, Schaefer et al., (1989) JACC 14, 472-480; Shreve et al., (1986) Magn. Reson. Med. 3, 336-340; Wolf, G L., (1984) Physiol. Chem. Phys. Med. NMR 16, 93-95; Wesbey et al., (1984) Physiol. Chem. Phys. Med. NMR 16, 145-155; Runge et al., (1984) Invest Radiol. 19, 408-415 for discussions on in vivo nuclear magnetic resonance imaging.)

An image can be generated in a method of the invention by computer assisted tomography (CAT), magnetic resonance spectroscopy (MRS) image, magnetic resonance imaging (MRI), positron emission tomography (PET), single-photon emission computed tomography (SPECT), or bioluminescence imaging (BLI) or equivalent.

Computer assisted tomography (CAT) and computerized axial tomography (CAT) systems and devices well known in the art can be utilized in the practice of the present invention. (See, for example, U.S. Pat. Nos. 6,151,377; 5,946,371; 5,446,799; 5,406,479; 5,208,581; 5,109,397). The invention may also utilize animal imaging modalities, such as MicroCAT.™ (ImTek, Inc.).

Magnetic resonance imaging (MRI) systems and devices well known in the art can be utilized in the practice of the present invention. In magnetic resonance methods and devices, a static magnetic field is applied to a tissue or a body in order to define an equilibrium axis of magnetic alignment in a region of interest. A radio frequency field is then applied to the region in a direction orthogonal to the static magnetic field direction to excite magnetic resonance in the region. The resulting radio frequency signals are then detected and processed, and the exciting radio frequency field is applied. The resulting signals are detected by radio-frequency coils that are placed adjacent to the tissue or area of the body of interest. (For a description of MRI methods and devices see, for example, U.S. Pat. Nos. 6,151,377; 6,144,202; 6,128,522; 6,127,825; 6,121,775; 6,119,032; 6,115,446; 6,111,410; 602,891; 5,555,251; 5,455,512; 5,450,010; 5,378,987; 5,214,382; 5,031,624; 5,207,222; 4,985,678; 4,906,931; 4,558,279). MRI and supporting devices are commercially available for example, from Bruker Medical GMBH; Caprius; Esaote Biomedica; Fonar; GE Medical Systems (GEMS); Hitachi Medical Systems America; Intermagnetics General Corporation; Lunar Corp.; MagneVu; Marconi Medicals; Philips Medical Systems; Shimadzu; Siemens; Toshiba America Medical Systems; including imaging systems, by, e.g., Silicon Graphics. The invention may also utilize animal imaging modalities such as micro-MRIs.

Positron emission tomography imaging (PET) systems and devices well known in the art can be utilized in the practice of the present invention. For example, a method of the invention may use the system designated Pet VI located at Brookhaven National Laboratory. For descriptions of PET systems and devices see, for example, U.S. Pat. Nos. 6,151,377; 6,072,177; 5,900,636; 5,608,221; 5,532,489; 5,272,343; 5,103,098. Animal imaging modalities such as micro-PETs (Corcorde Microsystems, Inc.) can also be used in the invention.

Single-photon emission computed tomography (SPECT) systems and devices well known in the art can be utilized in the practice of the present invention. (See, for example, U.S. Pat. Nos. 6,115,446; 6,072,177; 5,608,221; 5,600,145; 5,210,421; 5,103,098.) The methods of the invention may also utilize animal imaging modalities, such as micro-SPECTs.

Bioluminescence imaging includes bioluminescence, fluorescence or chemiluminescence or other photon detection systems and devices that are capable of detecting bioluminescence, fluorescence or chemiluminescence. Sensitive photon detection systems can be used to detect bioluminescent and fluorescent proteins externally; see, for example, Contag (2000) Neoplasia 2:41-52; Zhang (1994) Clin. Exp. Metastasis 12:87-92. The methods of the invention can be practiced using any such photon detection device, or variation or equivalent thereof, or in conjunction with any known photon detection methodology, including visual imaging. By way of example, an intensified charge-coupled device (ICCD) camera coupled to an image processor may be used in the present invention. (See, e.g., U.S. Pat. No. 5,650,135). Photon detection devices are also commercially available from Xenogen, Hamamatsue.

Kits

The invention also contemplates kits for carrying out the methods of the invention. Such kits typically comprise two or more components required for performing a diagnostic assay. Components include but are not limited to compounds, reagents, containers, and/or equipment.

The methods described herein may be performed by utilizing prepackaged diagnostic kits comprising at least one specific KLK9 nucleic acid or antibody described herein, which may be conveniently used, e.g., in clinical settings, to screen and diagnose patients and to screen and identify those individuals exhibiting a predisposition to developing a disorder.

In an embodiment, a container with a kit comprises a binding agent as described herein. By way of example, the kit may contain antibodies specific for hK9, antibodies against the anti-hK9 antibodies labelled with an enzyme; and a substrate for the enzyme. The kit may also contain microtiter plate wells, standards, assay diluent wash buffer, adhesive plate covers, and/or instructions for carrying out a method of the invention using the kit.

In an aspect of the invention, the kit includes an antibody or an antibody fragment which binds specifically to an epitope of a kallikrein 9, and means for detecting binding of the antibody to its epitope associated with tumor cells, either as concentrates (including lyophilized compositions), which may be further diluted prior to use or at the concentration of use, where the vials may include one or more dosages. Where the kits are intended for in vivo use, single dosages may be provided in sterilized containers, having the desired amount and concentration of agents. Containers that provide a formulation for direct use, usually do not require other reagents, as for example, where the kit contains a radiolabelled antibody preparation for in vivo imaging.

A kit may be designed to detect the level of nucleic acid molecules encoding hK9 in a sample. Such kits generally comprise at least one oligonucleotide probe or primer, as described herein, that hybridizes to a polynucleotide encoding a hK9 protein. Such an oligonucleotide may be used, for example, within a PCR or hybridization procedure. Additional components that may be present within the kits include a second oligonucleotide and/or a diagnostic reagent to facilitate detection of a polynucleotide encoding a hK9 protein.

Therapeutic Applications

hK9 is a target for ovarian cancer immunotherapy. Such immunotherapeutic methods include the use of antibody therapy, in vivo vaccines, and ex vivo immunotherapy approaches.

In one aspect, the invention provides hK9 antibodies that may be used systemically to treat ovarian cancer. Preferably antibodies are used that target the tumor cells but not the surrounding non-tumor cells and tissue. Thus, the invention provides a method of treating a patient susceptible to, or having a cancer that expresses hK9, comprising administering to the patient an effective amount of an antibody that binds specifically to hK9. In another aspect, the invention provides a method of inhibiting the growth of tumor cells expressing hK9, comprising administering to a patient an antibody which binds specifically to a hK9 in an amount effective to inhibit growth of the tumor cells. hK9 antibodies may also be used in a method for selectively inhibiting the growth of or killing a cell expressing hK9 comprising reacting a hK9 antibody immunoconjugate or immunotoxin with the cell in an amount sufficient to inhibit the growth of or kill the cell.

By way of example, unconjugated hK9 antibody may be introduced into a patient such that the antibody binds to hK9 expressing cancer cells and mediates growth inhibition of such cells (including the destruction thereof), and the tumor, by mechanisms which may include complement-mediated cytolysis, antibody-dependent cellular cytotoxicity, altering the physiologic function of hK9 and/or the inhibition of ligand binding or signal transduction pathways. In addition to unconjugated hK9 antibodies, hK9 antibodies conjugated to therapeutic agents (e.g. immunoconjugates) may also be used therapeutically to deliver the agent directly to hK9 expressing tumor cells and thereby destroy the tumor. Examples of such agents include abrin, ricin A, Pseudomonas exotoxin, or diphtheria toxin; proteins such as tumor necrosis factor, alpha-interferon, beta-interferon, nerve growth factor, platelet derived growth factor, tissue plasminogen activator; and biological response modifiers such as lymphokines, interleukin-1, interleukin-2, interleukin-6, granulocyte macrophage colony stimulating factor, granulocyte colony stimulating factor, or other growth factors.

Cancer immunotherapy using hK9 antibodies may utilize the various approaches that have been successfully employed for cancers, including but not limited to colon cancer (Arlen et al., 1998, Crit Rev Immunol 18: 133-138), multiple myeloma (Ozaki et al., 1997, Blood 90: 3179-3186; Tsunenati et al., 1997, Blood 90: 2437-2444), gastric cancer (Kasprzyk et al., 1992, Cancer Res 52: 2771-2776), B-cell lymphoma (Funakoshi et al., 1996, J Immunther Emphasis Tumor Immunol 19: 93-101), leukemia (Zhong et al., 1996, Leuk Res 20: 581-589), colorectal cancer (Moun et al., 1994, Cancer Res 54: 6160-6166); Velders et al., 1995, Cancer Res 55: 4398-4403), and breast cancer (Shepard et al., 1991, J Clin Immunol 11: 117-127).

In the practice of the method of the invention, anti-hK9 antibodies capable of inhibiting the growth of cancer cells expressing hK9 are administered in a therapeutically effective amount to cancer patients whose tumors express or overexpress hK9. The invention may provide a specific, effective and long-needed treatment for ovarian cancer. The antibody therapy methods of the invention may be combined with other therapies including chemotherapy and radiation.

Patients may be evaluated for the presence and level of hK9 expression and overexpression in tumors, preferably using immunohistochemical assessments of tumor tissue, quantitative hK9 imaging as described herein, or other techniques capable of reliably indicating the presence and degree of hK9 expression. Immunohistochemical analysis of tumor biopsies or surgical specimens may be employed for this purpose.

Anti-hK9 antibodies useful in treating cancer include those that are capable of initiating a potent immune response against the tumor and those that are capable of direct cytotoxicity. In this regard, anti-hK9 antibodies may elicit tumor cell lysis by either complement-mediated or antibody-dependent cell cytotoxicity (ADCC) mechanisms, both of which require an intact Fc portion of the immunoglobulin molecule for interaction with effector cell Fc receptor sites or complement proteins. In addition, anti-hK9 antibodies that exert a direct biological effect on tumor growth are useful in the practice of the invention. Such antibodies may not require the complete immunoglobulin to exert the effect. Potential mechanisms by which such directly cytotoxic antibodies may act include inhibition of cell growth, modulation of cellular differentiation, modulation of tumor angiogenesis factor profiles, and the induction of apoptosis. The mechanism by which a particular antibody exerts an anti-tumor effect may be evaluated using any number of in vitro assays designed to determine ADCC, antibody-dependent macrophage-mediated cytotoxicity (ADMMC), complement-mediated cell lysis, and others known in the art.

The anti-tumor activity of a particular anti-hK9 antibody, or combination of anti-hK9 antibodies, may be evaluated in vivo using a suitable animal model. Xenogenic cancer models, wherein human cancer explants or passaged xenograft tissues are introduced into immune compromised animals, such as nude or SCID mice, may be employed.

The methods of the invention contemplate the administration of single anti-hK9 antibodies as well as combinations, or “cocktails”, of different individual antibodies such as those recognizing different epitopes or other kallikreins. Such cocktails may have certain advantages inasmuch as they contain antibodies that bind to different epitopes or kallikreins and/or exploit different effector mechanisms or combine directly cytotoxic antibodies with antibodies that rely on immune effector functionality. Such antibodies in combination may exhibit synergistic therapeutic effects. In addition, the administration of anti-hK9 antibodies may be combined with other therapeutic agents, including but not limited to chemotherapeutic agents, androgen-blockers, and immune modulators (e.g., IL2, GM-CSF). The anti-hK9 antibodies may be administered in their “naked” or unconjugated form, or may have therapeutic agents conjugated to them.

The anti-hK9 antibodies used in the practice of the method of the invention may be formulated into pharmaceutical compositions comprising a carrier suitable for the desired delivery method. Suitable carriers include any material which when combined with the antibodies retains the anti-tumor function of the antibody and is non-reactive with the subject's immune systems. Examples include any of a number of standard pharmaceutical carriers such as sterile phosphate buffered saline solutions, bacteriostatic water, and the like (see, generally, Remington's Pharmaceutical Sciences 16.sup.th Edition, A. Osal., Ed., 1980).

Anti-hK9 antibody formulations may be administered via any route capable of delivering the antibodies to the tumor site. Routes of administration include, but are not limited to, intravenous, intraperitoneal, intramuscular, intratumor, intradermal, and the like. Preferably, the route of administration is by intravenous injection. Anti-hK9 antibody preparations may be lyophilized and stored as a sterile powder, preferably under vacuum, and then reconstituted in bacteriostatic water containing, for example, benzyl alcohol preservative, or in sterile water prior to injection.

Treatment will generally involve the repeated administration of the antibody preparation via an acceptable route of administration such as intravenous injection (IV), at an effective dose. Dosages will depend upon various factors generally appreciated by those of skill in the art, including the type of cancer and the severity, grade, or stage of the cancer, the binding affinity and half life of the antibodies used, the degree of hK9 expression in the patient, the extent of circulating hK9 antigen, the desired steady-state antibody concentration level, frequency of treatment, and the influence of any chemotherapeutic agents used in combination with the treatment method of the invention. Daily doses may range from about 0.1 to 100 mg/kg. Doses in the range of 10-500 mg antibodies per week may be effective and well tolerated, although even higher weekly doses may be appropriate and/or well tolerated. A determining factor in defining the appropriate dose is the amount of a particular antibody necessary to be therapeutically effective in a particular context. Repeated administrations may be required to achieve tumor inhibition or regression. Direct administration of hK9 antibodies is also possible and may have advantages in certain situations.

Patients may be evaluated for serum hK9 in order to assist in the determination of the most effective dosing regimen and related factors. The hK9 assay methods described herein, or similar assays, may be used for quantitating circulating hK9 levels in patients prior to treatment. Such assays may also be used for monitoring throughout therapy, and may be useful to gauge therapeutic success in combination with evaluating other parameters such as serum hK9 levels.

The invention further provides vaccines formulated to contain a hK9 protein or fragment thereof. In an embodiment, the invention provides a method of vaccinating an individual against hK9 comprising the step of inoculating the individual with a hK9 protein or fragment thereof that lacks activity, wherein the inoculation elicits an immune response in the individual thereby vaccinating the individual against hK9.

The use in anti-cancer therapy of a tumor antigen in a vaccine for generating humoral and cell-mediated immunity is well known and, for example, has been employed in prostate cancer using human PSMA and rodent PAP immunogens (Hodge et al., 1995, Int. J. Cancer 63: 231-237; Fong et al., 1997, J. Immunol. 159: 3113-3117). These methods can be practiced by employing a hK9 protein, or fragment thereof, or a hK9-encoding nucleic acid molecule and recombinant vectors capable of expressing and appropriately presenting the hK9 immunogen.

By way of example, viral gene delivery systems may be used to deliver hK9 encoding nucleic acid molecule. Various viral gene delivery systems which can be used in the practice of this aspect of the invention include, but are not limited to, vaccinia, fowlpox, canarypox, adenovirus, influenza, poliovirus, adeno-associated virus, lentivirus, and sindbus virus (Restifo, 1996, Curr. Opin. Immunol. 8: 658-663). Non-viral delivery systems may also be employed by using naked DNA encoding a hK9 protein or fragment thereof introduced into the patient (e.g., intramuscularly) to induce an anti-tumor response.

Various ex vivo strategies may also be employed. One approach involves the use of cells to present hK9 antigen to a patient's immune system. For example, autologous dendritic cells which express MHC class I and II, may be pulsed with hK9 or peptides thereof that are capable of binding to MHC molecules, to thereby stimulate cancer (e.g. ovarian cancer) patients' immune systems (See, for example, Tjoa et al., 1996, Prostate 28: 65-69; Murphy et al., 1996, Prostate 29: 371-380).

Anti-idiotypic anti-hK9 antibodies can also be used in anti-cancer therapy as a vaccine for inducing an immune response to cells expressing a hK9 protein. The generation of anti-idiotypic antibodies is well known in the art and can readily be adapted to generate anti-idiotypic anti-hK9 antibodies that mimic an epitope on a hK9 protein (see, for example, Wagner et al., 1997, Hybridoma 16: 3340; Foon et al., 1995, J Clin Invest 96: 334-342; Herlyn et al., 1996, Cancer Immunol Immunother 43: 65-76). Such an antibody can be used in anti-idiotypic therapy as presently practiced with other anti-idiotypic antibodies directed against tumor antigens.

Genetic immunization methods may be utilized to generate prophylactic or therapeutic humoral and cellular immune responses directed against cancer cells expressing hK9. Using the hK9 encoding DNA molecules, constructs comprising DNA encoding a hK9 protein/immunogen and appropriate regulatory sequences may be injected directly into muscle or skin of an individual, such that the cells of the muscle or skin take-up the construct and express the encoded hK9 protein/immunogen. The hK9 protein/immunogen may be expressed as a cell surface protein or be secreted. Expression of the hK9 protein/immunogen results in the generation of prophylactic or therapeutic humoral and cellular immunity against the cancer. Various prophylactic and therapeutic genetic immunization techniques known in the art may be used.

The invention further provides methods for inhibiting cellular activity (e.g., cell proliferation, activation, or propagation) of a cell expressing hK9. This method comprises reacting immunoconjugates of the invention (e.g., a heterogeneous or homogenous mixture) with the cell so that the hK9 proteins form a complex with the immunoconjugates. A subject with a neoplastic or preneoplastic condition can be treated when the inhibition of cellular activity results in cell death.

In another aspect, the invention provides methods for selectively inhibiting a cell expressing hK9 by reacting any one or a combination of immunoconjugates of the invention with the cell in an amount sufficient to inhibit the cell. Amounts include those that are sufficient to kill the cell or sufficient to inhibit cell growth or proliferation.

Vectors derived from retroviruses, adenovirus, herpes or vaccinia viruses, or from various bacterial plasmids, may be used to deliver nucleic acid molecules encoding hK9 to a targeted organ, tissue, or cell population. Methods well known to those skilled in the art may be used to construct recombinant vectors that will express antisense nucleic acid molecules for hK9. (See, for example, the techniques described in Sambrook et al (supra) and Ausubel et al (supra)).

Methods for introducing vectors into cells or tissues include those methods discussed herein and which are suitable for in vivo, in vitro and ex vivo therapy. For ex vivo therapy, vectors may be introduced into stem cells obtained from a patient and clonally propagated for autologous transplant into the same patient (See U.S. Pat. Nos. 5,399,493 and 5,437,994). Delivery by transfection and by liposome are well known in the art.

Genes encoding a hK9 protein can be turned off by transfecting a cell or tissue with vectors that express high levels of a desired hK9-encoding fragment. Such constructs can inundate cells with untranslatable sense or antisense sequences. Even in the absence of integration into the DNA, such vectors may continue to transcribe RNA molecules until all copies are disabled by endogenous nucleases.

Modifications of gene expression can be obtained by designing antisense molecules, DNA, RNA or PNA, to the regulatory regions of a gene encoding a hK9, i.e., the promoters, enhancers, and introns. Preferably, oligonucleotides are derived from the transcription initiation site, eg, between −10 and +10 regions of the leader sequence. The antisense molecules may also be designed so that they block translation of mRNA by preventing the transcript from binding to ribosomes. Inhibition may also be achieved using “triple helix” base-pairing methodology. Triple helix pairing compromises the ability of the double helix to open sufficiently for the binding of polymerases, transcription factors, or regulatory molecules. Therapeutic advances using triplex DNA were reviewed by Gee J E et al (In: Huber B E and B I Carr (1994) Molecular and Immunologic Approaches, Futura Publishing Co, Mt Kisco N.Y.).

Ribozymes are enzymatic RNA molecules that catalyze the specific cleavage of RNA. Ribozymes act by sequence-specific hybridization of the ribozyme molecule to complementary target RNA, followed by endonucleolytic cleavage. The invention therefore contemplates engineered hammerhead motif ribozyme molecules that can specifically and efficiently catalyze endonucleolytic cleavage of sequences encoding a hK9 protein.

Specific ribozyme cleavage sites within any potential RNA target may initially be identified by scanning the target molecule for ribozyme cleavage sites that include the following sequences, GUA, GUU and GUC. Once the sites are identified, short RNA sequences of between 15 and 20 ribonucleotides corresponding to the region of the target gene containing the cleavage site may be evaluated for secondary structural features which may render the oligonucleotide inoperable. The suitability of candidate targets may also be determined by testing accessibility to hybridization with complementary oligonucleotides using ribonuclease protection assays.

hK9 proteins and nucleic acids encoding the protein, and fragments thereof, may be used in the treatment of ovarian cancer in a subject. The proteins or nucleic acids may be formulated into compositions for administration to subjects suffering from ovarian cancer. Therefore, the present invention also relates to a composition comprising a hK9 protein or a nucleic acid encoding the protein, or a fragment thereof, and a pharmaceutically acceptable carrier, excipient or diluent. A method for treating or preventing ovarian cancer in a subject is also provided comprising administering to a patient in need thereof, a hK9 protein or a nucleic acid encoding the protein, or a composition of the invention.

The active substance may be administered in a convenient manner such as by injection (subcutaneous, intravenous, etc.), oral administration, inhalation, transdermal application, or rectal administration. Depending on the route of administration, the active substance may be coated in a material to protect the substance from the action of enzymes, acids and other natural conditions that may inactivate the substance.

The compositions described herein can be prepared by per se known methods for the preparation of pharmaceutically acceptable compositions which can be administered to subjects, such that an effective quantity of the active substance is combined in a mixture with a pharmaceutically acceptable vehicle. Suitable vehicles are described, for example, in Remington's Pharmaceutical Sciences (Remington's Pharmaceutical Sciences, Mack Publishing Company, Easton, Pa., USA 1985). On this basis, the compositions include, albeit not exclusively, solutions of the active substances in association with one or more pharmaceutically acceptable vehicles or diluents, and contained in buffered solutions with a suitable pH and iso-osmotic with the physiological fluids.

The compositions are indicated as therapeutic agents either alone or in conjunction with other therapeutic agents or other forms of treatment (e.g. chemotherapy or radiotherapy). The compositions of the invention may be administered concurrently, separately, or sequentially with other therapeutic agents or therapies.

The following non-limiting examples are illustrative of the present invention:

### EXAMPLE 1

Materials and Methods

Study Population

Included in this study were tumor specimens from 168 consecutive patients undergoing surgical treatment for epithelial ovarian carcinoma at the Department of Gynecological Oncology at the University of Turin, Turin Italy. Selection criteria included confirmation of diagnosis by histopathology. No patient received any treatment before surgery.

Patient ages ranged from 25 to 82 with a median of 59 years. Residual tumors after surgery sizes ranged from 0 to 9 cm, with a median of 2 cm. Follow-up information (median follow-up period 62 months) was available from 166 patients, among whom 91 (55%) had relapsed and 56 (34%) died. With respect to histological type, 82 tumors were serous papillary, 31 were endometrioid, 27 were undifferentiated, 13 were mucinous, and 14 were clear cell. The size of the residual tumors ranged from 0 to 9 cm, with a median of 1.0 cm.

Classification of histological types followed the World Health Organization criteria (37). All patients were staged according to the International Federation of Gynecology and Obstetrics staging system (38). Grading information was available for 167 patients; 59 (35%) had grade 1 or 2 and 108 (65%) had grade 3 ovarian carcinoma. Grading was established for each ovarian tumor according to the criteria of Day et al. (39). All patients were treated with postoperative platinum chemotherapy. The first-line chemotherapy regimens included cisplatin in 94 (56%) patients, carboplatin in 50 (30%), cyclophosphamide in 69 (41%), doxorubicin in 12 (7%), epirubicin in 20 (12%), paclitaxel in 27 (16%), and methotrexate in 2 (1%). Grade 1 and stage I patients received no further treatment. Response to chemotherapy was assessed as follows: complete response was defined as a resolution of all evidence of disease for at least 1 month; a decrease (lasting at least 1 month) of at least 50% in the diameters of all measurable lesions without the development of new lesions was termed partial response. Stable disease was defined as a decrease of less than 25% in the product of the diameters of all measurable lesions, an increase of at least 25% was termed as a progressive disease. Investigations were performed in accordance with the Helsinki declaration and were approved by the Institute of Obstetrics and Gynecology, Turin, Italy. Tumor specimens were snap-frozen in liquid nitrogen immediately after surgery. Histological examination, performed during intra-surgery frozen section analysis, allowed representative portions of each tumor containing >80% tumor cells to be selected for storage until analysis.

Total RNA Extraction and cDNA Synthesis

Samples were shipped and stored at −80° C. They were then minced with a scalpel, on dry ice, and transferred immediately to 2 ml polypropylene tubes. They were then homogenized and total RNA was extracted using Trizol™ reagent (Gibco BRL, Gaithersburg, Md.) following the manufacturer's instructions. The concentration and purity of RNA were determined spectrophotometrically. 2 μg of total RNA was reverse-transcribed into first strand cDNA using the Superscript™ preamplification system (Gibco BRL). The final volume was 20 μl.

Quantitative Real-Time RT-PCR Analysis

Based on the published genomic sequence of KLK9 (GenBank accession # AF135026), two gene-specific primers were designed (L2-3:5′ CAA GAC CCC CCT GGA TGT GG 3′ [SEQ ID NO. 4] and 5L2: 5′ AGT TTT CAG AGT CCG TCT CGG 3′ [SEQ ID NO.5]). These primers spanned more than 2 exons to avoid contamination by genomic DNA.

Real-time monitoring of PCR reactions was performed using the LightCycler™ system (Roche Molecular Systems, Indianapolis, Ind., USA) and the SYBR green I dye, which binds preferentially to double stranded DNA. Fluorescence signals, which are proportional to the concentration of the PCR product, are measured at the end of each cycle and immediately displayed on a computer screen, permitting real time monitoring of the PCR reaction (40). The reaction is characterized by the point during cycling, when amplification of PCR products is first detected, rather than the amount of PCR product accumulated after a fixed number of cycles. The higher the starting quantity of the template, the earlier a significant increase in fluorescence is observed (41). The threshold cycle is defined as the fractional cycle number at which fluorescence passes a fixed threshold above baseline (42).

Endogenous Control

For each sample, the amount of the target and of an endogenous control (β actin, a housekeeping gene) were determined using a calibration curve (see below). The amount of the target molecule was then divided by the amount of the endogenous reference, to obtain a normalized target value (41).

Calibration Curves

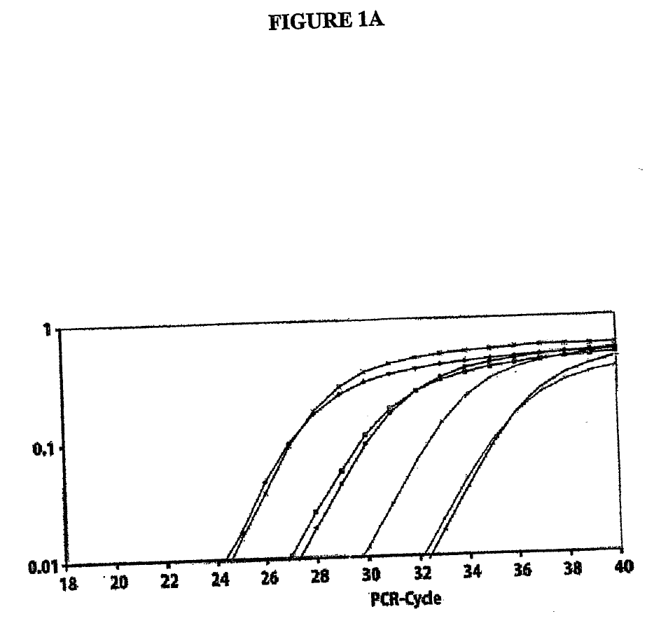

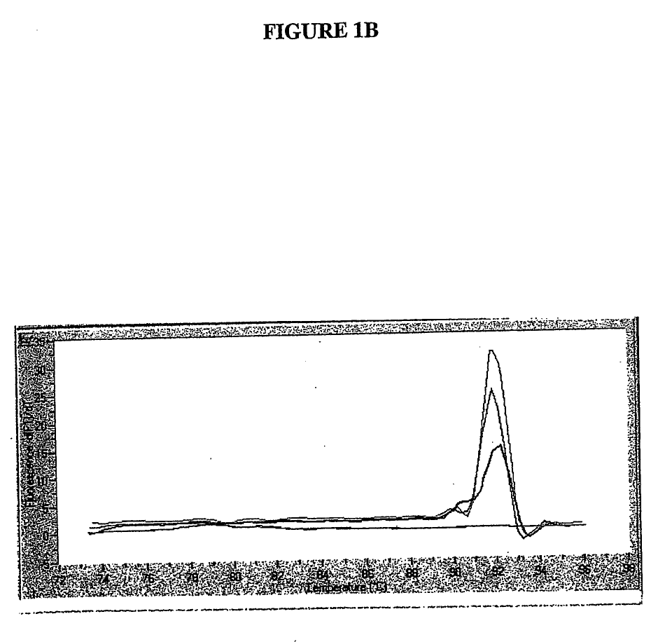

Separate calibration (standard) curves for actin and KLK9 were constructed using serial dilutions of total cDNA from healthy human ovarian tissue, purchased from Clontech, Palo Alto, Calif., as described previously (41, 43). The standard curve calibrators were included in each run. The Lightcycler™ software automatically calculates the standard curve by plotting the starting dilution of each standard sample versus the threshold cycle, and the sample concentrations were then calculated accordingly (FIG. 1). Standards for both KLK9 and actin RNAs were defined to contain an arbitrary starting concentration, and serial dilutions (with concentrations defined according to the dilution factor) were used to construct the standard curve.

PCR Amplification

The PCR reaction was carried out on the LightCycler™ system. For each run, a master mixture was prepared on ice, containing 1 μl of cDNA, 2 μl of LC DNA Master SYBR Green I mix, 50 ng of primers and 1.2 μl of 25 mM MgCl₂. The final volume was adjusted with H₂O to 20 μl. After the reaction mixture was loaded into a glass capillary tube, the cycling conditions were carried out as follows: initial denaturation at 94° C. for 10 minutes, followed by 45 cycles of denaturation at 94° C. for 0 s, annealing at 55° C. for 10 s, and extension at 72° C. for 30 s. The temperature transition rate was set at 20° C. per second. Fluorescent product was measured by a single acquisition mode at 86° C. after each cycle.

Melting Curve

For distinguishing specific from non-specific products and primer dimers, a melting curve was obtained after amplification by holding the temperature at 70° C. for 30 s, followed by a gradual increase in temperature to 99° C. at a rate of 0.1° C./s, with the signal acquisition mode set at step, as described (44) (FIG. 1). To verify the melting curve results, representative samples of the PCR products were run on 1.5% agarose gels, purified, and cloned into the pCR 2.1-TOPO vector (Invitrogen, Carlsbad, Calif., USA) according to the manufacturer's instructions. The inserts were sequenced using vector-specific primers, with an automated DNA sequencer.

Statistical Analysis

Associations between clinicopathological parameters such as stage, grade, histotype, and residual tumor, and KLK9 expression were analyzed by the chi-square test or the Fisher's Exact Test, when appropriate. For survival analysis, two different end points, cancer relapse (either local recurrence or distant metastasis) and death, were used to calculate progression free and overall survival, respectively. Progression free survival was defined as the time interval between the date of surgery and the date of identification of recurrent or metastatic disease. Overall survival was defined as the time interval between the date of surgery and the date of death.

The Cox univariate and multivariate proportional hazard regression model (45) was used to evaluate the hazard ratio (relative risk of relapse or death in the KLK9-positive group). In the multivariate analysis, the models were adjusted for KLK9 expression, clinical stage, histologic grade, residual tumor and age.

Kaplan-Meier survival curves (46) were constructed for KLK9-positive and KLK9-negative patients. For further analysis, patients were divided into two groups either by the tumor grade (grade 1-2 vs. grade 3), tumor stage (stage I-II vs stage III-IV), or by the success of debulking (optimal vs. suboptimal debulking group). In each category, survival rates (disease-free survival and overall survival) were compared between KLK9-positive arid KLK9-negative groups. The differences between the survival curves between groups were tested for statistical significance by the log rank test (47).

Immunohistochemistry

Rabbit polyclonal antibody was raised against hK9 peptide sequence: N₂H-CPHPGFNKDLSANDHN-CONH₂ [SEQ ID NO. 6] according to standard procedures. Immunohistochemical staining for hK9 was performed according to a standard immunoperoxidase method. Briefly, paraffin-embedded tissue sections (4 μm) were fixed and dewaxed. Endogenous peroxidase activity was blocked with 3% aqueous hydrogen peroxide for 15 min. Sections were then treated with 0.4% pepsin at pH 2.0 for 5 min at 42° C. and blocked with 20% protein blocker (Signet Labs) for 10 min. The primary antibody was then added at 1:6000 for 1 hour at room temperature. After washing, biotinylated anti-rabbit antibody (Signet Labs) was added, diluted 4-fold in antibody dilution buffer (Dako), following incubation and washing, streptavidin-tagged horseradish peroxidase was added for 30 min at room temperature. After washing, detection was achieved with amino ethyl carbazol (AEC) for 5-10 min. The slides were then counterstained with hematoxylin and then mounted with cover slips.

Cell Lines and Hormonal Stimulation Experiments

The epithelial ovarian cancer cell line BG-1 and breast cancer cell lines BT-474 and T-47D and MCF-7 line were purchased from the American Type Culture Collection (ATCC), Rockville, Md. Cells were cultured in RPMI media (Gibco BRL, Gaithersburg, Md.) supplemented with glutamine (200 mmol/L), bovine insulin (10 mg/L), fetal bovine serum (10%), antibiotics and antimycotics, in plastic flasks, to near confluency. The cells were then aliquoted into 24-well tissue culture plates and cultured to 50% confluency. 24 hours before the experiments, the culture media were changed into phenol red-free media containing 10% charcoal-stripped fetal bovine serum. For stimulation experiments, various steroid hormones dissolved in 100% ethanol were added into the culture media at a final concentration of 10⁻⁸ M. Cells stimulated with 100% ethanol were included as controls. The cells were cultured for 24 hours, then harvested for mRNA extraction.

Results

KLK9 Expression and Relation to Other Variables

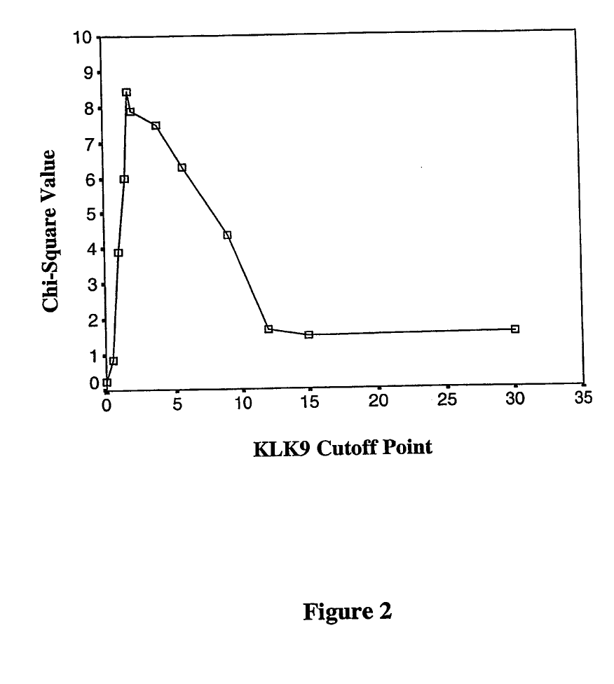

First, an optimal cutoff value was defined by χ² analysis, based on the ability of KLK9 values to predict the OS of the study population. As shown in FIG. 2, a value of 1.84 (this is a unitless ratio) was shown to be the optimal cutoff (χ²=8.54, p=0.003). This cutoff (54^{th} percentile) identifies 46% of patients as being KLK9 positive.

Table 1 depicts the distribution of KLK9 expression (positive or negative) in ovarian tumor tissues, in relation to clinical stage, grade, histological type, size of residual tumor, menopausal status and chemotherapy response. KLK9 expression was significantly higher in patients with early stages (I or II) compared with advanced stages (III or IV) (P=0.044) and in patients with optimal debulking (p=0.019). Also, slightly higher percentage of patients with positive KLK9 expression was found to have grade 1 or 2 (46%) compared to grade 3 (44%), however, this difference was not statistically significant (p=0.49). Also, KLK9 expression was higher (although not statistically significant) in patients with complete or partial response to chemotherapy, compared to those with no response or progression of the disease (46% vs 37%, respectively) and in patients with no residual tumors (56%) compared to those with residual tumors (38%). On the other hand, no significant associations were found between KLK9 expression and different histological types, or menopausal status.

Survival Analysis

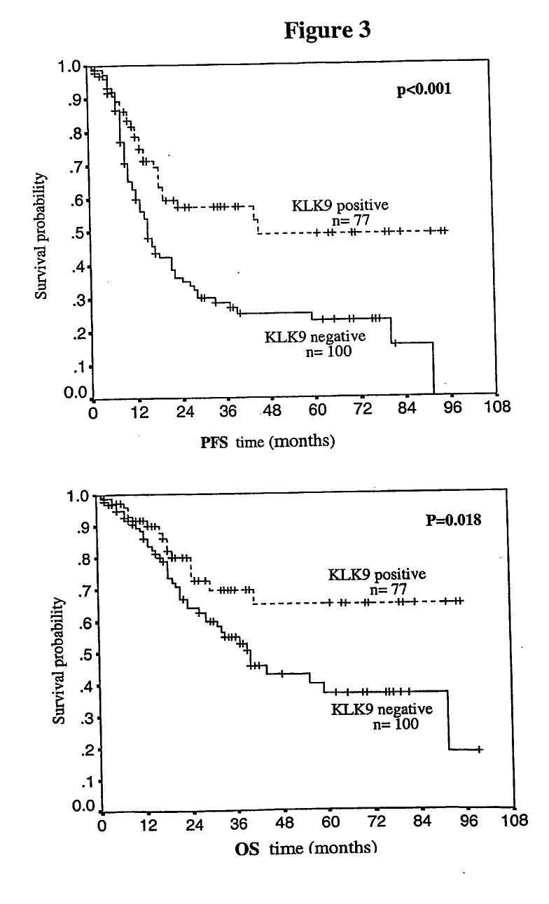

The strength of the associations between each individual predictor and progression-free or overall survival are shown in the univariate analysis in Table 2. Stage of disease, histological grade and residual tumor size showed a strong association with cancer relapse and death (p<0.001). KLK9 expression was also found to be a significant predictor of progression-free (PFS) and overall (OS) survival (hazard ratio of 0.45 and 0.49 and p value of <0.001 and 0.019, respectively). Kaplan-Meier survival curves (FIG. 3) also demonstrate that patients with KLK9-positive tumors have substantially longer PFS and OS (p<0.001, p=0.016, respectively) compared to those who are KLK9-negative.

When all the predictors were included in the Cox model (multivariate analysis, Table 2), the stage of disease and residual tumor size retained their prognostic significance. KLK9 expression retained its prognostic significance for PFS, but not the OS, (hazard ratio of 0.58 and 0.71 and p value of 0.025 and 0.28 for the PFS and OS, respectively).

When the Cox proportional hazard regression analysis was applied to subgroups of patients (Table 3), KLK9 expression was found to be a significant predictor of progression-free survival in the subgroup of patients with grade 1 or 2 (HR=0.13, p=0.0015) (Table 3), Stage I or II (HR=0.099, p=0.045) and those with optimal debulking success (HR=0.26, p=0.012). After adjusting for other known prognostic variables, KLK9 retained its independent prognostic value in all these subgroups of patients. With respect to the overall survival, KLK9 expression was a favourable prognostic marker for the subgroup of patients with grade 1 or 2 tumors, and retained its independent prognostic value after adjusting for other known prognostic variables (adjusted HR=0.20, p=0.038) (Table 3).

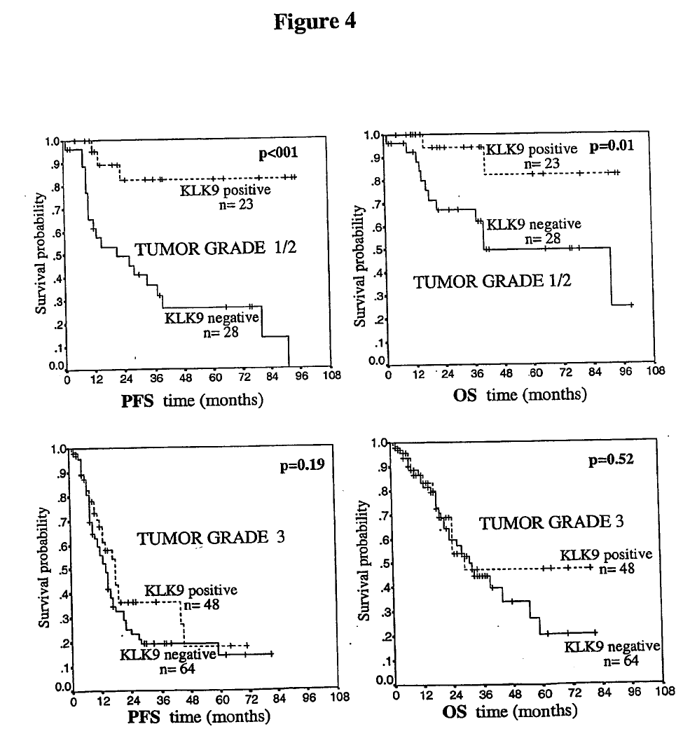

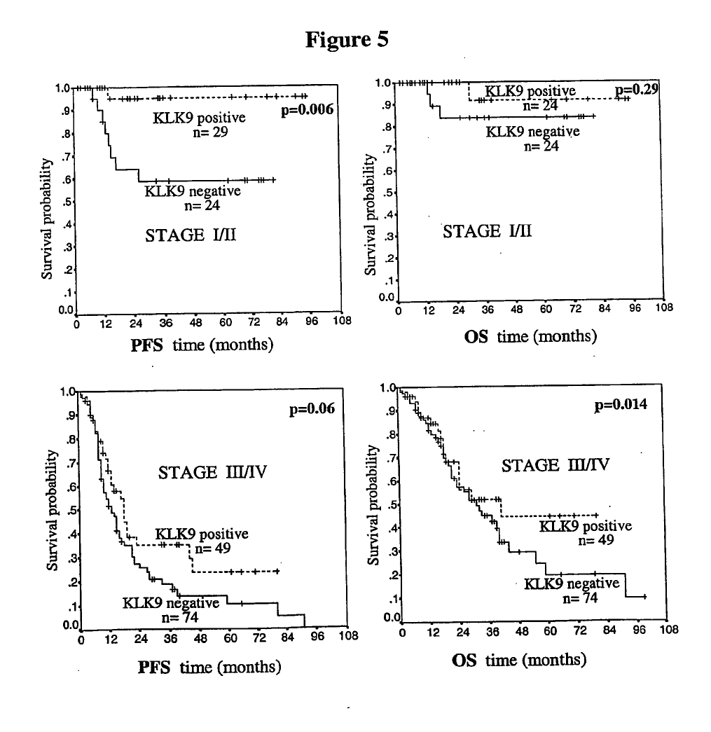

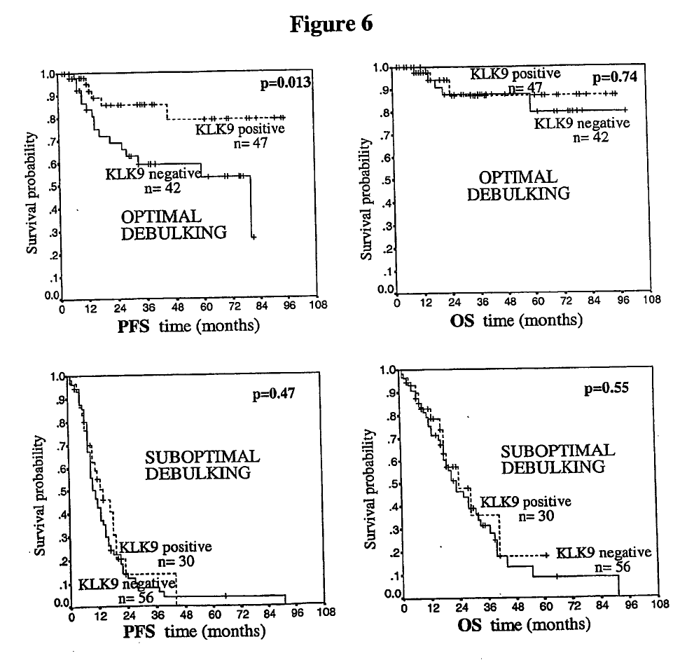

The same results were also demonstrated by the Kaplan-Meier curves, where KLK9 was found to be an independent favourable prognostic marker for PFS and OS (p<0.001 and 0.016, respectively). Shown in FIG. 4 are the progression-free and overall survival curves for cancer patients with histological grades 1-2 or 3. Patients with KLK9-positive tumors had substantially longer progression-free and overall survival than did patients with KLK9-negative tumors (P<0.001 and 0.021, respectively). These differences were not seen in patients with grade 3 tumors. With respect to stage of the disease, KLK9-positive patients in stage I or II have a significantly better PFS (p=0.007) but not OS (FIG. 5). Similarly, patients with KLK9-positive tumours who had undergone optimal debulking had a higher probability of PFS (but not OS) than did patients who had KLK9-negative tumors (p=0.013) (FIG. 6). No differences in the progression-free or overall survival were observed when surgical debulking was suboptimal (FIG. 6).

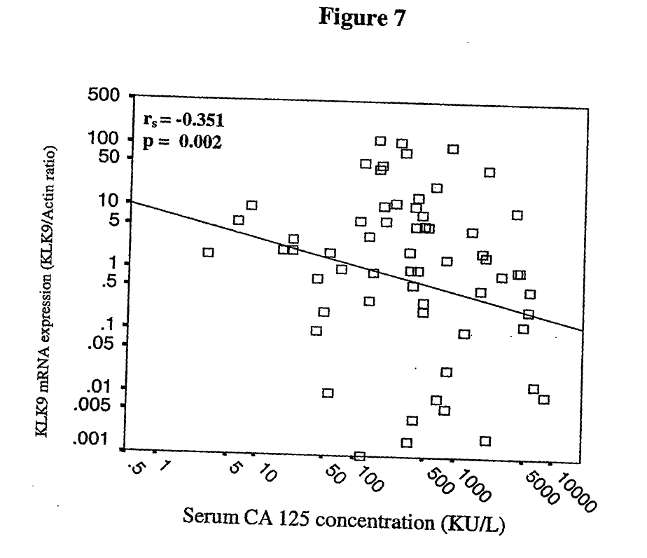

A weak negative correlation was found between the expression levels of serum CA125 and KLK9 mRNA levels (rₛ=0.350, p=0.002) (FIG. 7).

Immunohistochemical Localization of hK9

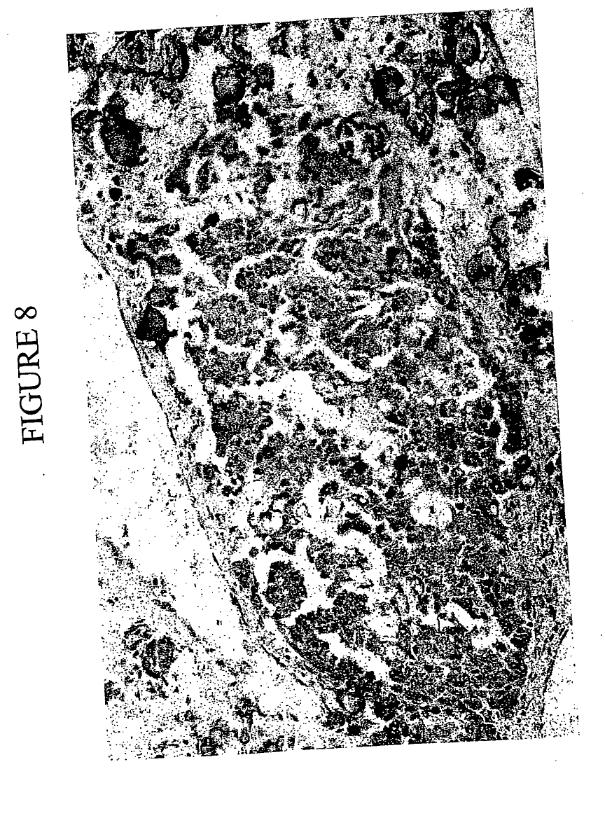

As shown in FIG. 8, hK9 is seen in cytoplasm, but not the nuclei of the epithelial cells of normal ovarian and ovarian cancer tissues, confirming the epithelial origin of the protein and the previous reports indicating that the protein is a secreted protein. These results are consistent with previous results for other kallikrein proteins which were also localized in the cytoplasm of the epithelial cells.

Hormonal Regulation of hK9

KLK9 expression was studied in an BG-1 epithelial ovarian cell line and the BT-474, T47-D and MCF-7 breast cancer cell lines. KLK9 was found to be unregulated by steroid hormones, particularly estrogens and progestins. Higher expression levels were obtained 48 hours after hormonal stimulation. No significant changes in KLK9 level were seen in the receptor-negative BT-20 cell lines.

Discussion

Population screening is a milestone for improving ovarian cancer prognosis. CA125 has limitations as a single marker, since its levels are elevated in only about half of women with stage I ovarian cancer. The development of new biomarkers for ovarian cancer may help to improve the diagnostic/prognostic power of CA125 (10-11). Although newly identified markers for ovarian cancer might also not be sufficient alone, the development of a panel of markers that can be used together, in multiparametric strategies, might be one solution (48). Jacobs et al (49) recently reported the first study with annual multimodal screening for three years. KLK9, along with a few other newly identified kallikreins, may be good candidates for this application (31, 32).

A recent study suggested that CA125 could be used for prediction of optimal primary tumor cytoreduction, but only in stage III tumors (8). Since KLK9 expression levels are significantly different in patients with optimal and suboptimal cytoreduction, and in patients with early and late stages of the disease (Table 3 and FIGS. 5,6), it might also be tested for such applications. In addition, the role of CA125 in follow up and prediction of prognosis is uncertain (7). KLK9, being a favorable prognostic factor (FIG. 3), may find applicability in this regard.

KLK9 is a favorable prognostic factor in ovarian cancer. The enzymatic activity of hK9 might initiate or terminate certain biological events, e.g., the onset of angiogenesis, activation or inactivation of growth factors, receptors, cytokines etc. Another closely related kallikrein, hK3 (PSA), has antiangiogenic activity, and this activity may be related to its action as a serine protease (51). This study suggested also that other members of the kallikrein multigene family should be evaluated for potential antiangiogenic action. Other studies suggested that PSA inhibits growth of MCF-7 breast cancer cell lines and prolongs the doubling time of PC-3 prostate cancer cell lines (52, 53).

KLK9 is up-regulated by steroid hormones, primarily progesterone, and estrogen. In this study, KLK9 was found to be a favorable prognostic factor for ovarian cancer. Ovarian cancer is one of the endocrine-related malignancies (54), and oral contraceptive pill administration decreases the risk of ovarian cancer (1). Furthermore, the growth of ovarian carcinoma cell lines is sensitive to estrogen (55). Progesterone promotes cell differentiation and apoptosis, and it has been shown to inhibit DNA synthesis and cell division (56). Recent studies supported the favorable prognostic value of progesterone receptor (PR) and its level of expression in ovarian cancer, and indicated that PR-negative status is more abundant in grade 3 ovarian tumors (54, 57). Taken together, these data suggest that KLK9 might be a candidate downstream target through which progestins and estrogens are involved in the pathogenesis of ovarian cancer.

KLK9 expression levels are negatively correlated with serum CA125 concentration (FIG. 7), in agreement with previous studies showing that higher CA125 levels are associated with poor prognosis in ovarian cancer (58). High CA125 expression levels were associated with serous and endometroid tumors (58). Here, equal levels of KLK9 expression were found in serous and non-serous tumors (45% vs 42%, p=0.39) (Table 1). This can be utilized for assessing prognosis, in the subgroup of patients with non-serous ovarian cancer, where CA125 is not usually informative.

In conclusion, higher KLK9 expression has been associated with favorable prognostic value in ovarian cancer.

While the present invention has been described with reference to what are presently considered to be the preferred examples, it is to be understood that the invention is not limited to the disclosed examples. To the contrary, the invention is intended to cover various modifications and equivalent arrangements included within the spirit and scope of the appended claims.

All publications, patents and patent applications are herein incorporated by reference in their entirety to the same extent as if each individual publication, patent or patent application was specifically and individually indicated to be incorporated by reference in its entirety.

Below full citations are set out for the references referred to in the specification.

TABLE 1
Relationship between KLK9 status and other
variables in 182 ovarian cancer patients.

| | | No. of patients (%) | | |
|---|---|---|---|---|
| Variable | Patients | KLK9 negative | KLK9 positive | P value |
| Stage | | | | |
| I/II | 53 | 24 (45.3) | 29 (54.7) | |
| III/IV | 126 | 77 (61.1) | 49 (38.8) | 0.037^{b} |
| x | 3 | | | |
| Grade | | | | |
| G1/G2 | 61 | 33 (54.1) | 28 (45.9) | |
| G3 | 115 | 66 (57.4) | 49 (42.6) | 0.39^{a} |
| x | 5 | | | |
| Histotype | | | | |
| Serous | 82 | 45 (54.9) | 37 (45.1) | 0.39^{b} |
| Others | 100 | 58 (58.0) | 42 (42.0) | |
| Residual tumor (cm) | | | | |
| 0 | 75 | 35 (46.7) | 40 (53.3) | |
| 1-2 | 32 | 19 (59.4) | 13 (40.6) | 0.075^{a} |
| >2 | 69 | 45 (65.2) | 24 (34.8) | |
| x | 8 | | | |
| Debulking success^{c} | | | | |
| OD | 82 | 42 (47.2) | 47 (52.8) | 0.011^{b} |
| SO | 87 | 57 (65.5) | 30 (34.5) | |
| x | 6 | | | |
| Menopause | | | | |
| Pre/peri | 59 | 37 (62.7) | 22 (37.3) | 0.16^{b} |
| Post | 123 | 66 (53.6) | 57 (46.4) | |
| Response to CTX^{d} | | | | |
| CR/PR | 151 | 83 (55.0) | 68 (45.0) | 0.19^{b} |
| NC/PD | 19 | 13 (68.4) | 6 (31.6) | |
| NE | 12 | | | |

^{a}χ² test.
^{b}Fisher's Exact Test
^{c}OD; Optimal debulking (0-1 cm), SO; Suboptimal debulking (>1 cm)
^{d}CTX; chemotherapy, NC; no change, PD; progressive disease, CR; complete response, PR; partial response, NE; not evaluated.
x. Status unknown.

TABLE 2
Univariate and Multivariate Analysis of KLK9 expression,
and relation to progression-free and overall survival.

| | Progression-free survival | | | Overall survival | | |
|---|---|---|---|---|---|---|
| Variable | HR^{a} | 95% CI^{b} | P value | HR^{a} | 95% CI^{b} | P value |
| | Univariate analysis | | | | | |
| KLK9 | | | | | | |
| Negative | 1.00 | | | 1.00 | | |
| Positive | 0.46 | 0.29-0.72 | <0.001 | 0.51 | 0.29-0.90 | 0.021 |
| As a | 0.99 | 0.98-1.00 | 0.13 | 0.97 | 0.95-1.00 | 0.065 |
| continuous | | | | | | |
| variable | | | | | | |
| Stage of | 2.79 | 2.06-3.79 | <0.001 | 3.07 | 2.05-4.60 | <0.001 |
| disease | | | | | | |
| (ordinal) | | | | | | |
| Grading | 2.23 | 1.63-3.07 | <0.001 | 2.34 | 1.53-3.58 | <0.001 |
| (ordinal) | | | | | | |
| Residual tumor | 1.27 | 1.21-1.34 | <0.001 | 1.31 | 1.21-1.41 | <0.001 |
| (ordinal) | | | | | | |
| Histologic | 0.68 | 0.46-1.00 | 0.055 | 0.78 | 0.48-1.29 | 0.34 |
| type^{c} | | | | | | |
| Age | 1.01 | 0.99-1.03 | 0.14 | 1.01 | 0.99-1.03 | 0.15 |
| | Multivariate analysis | | | | | |
| KLK9 | | | | | | |
| Negative | 1.00 | | | 1.00 | | |
| Positive | 0.63 | 0.40-0.98 | 0.037 | 0.69 | 0.38-1.26 | 0.23 |
| As a | 0.99 | 0.98-1.00 | 0.12 | 0.98 | 0.95-1.00 | 0.071 |
| continuous | | | | | | |
| variable | | | | | | |
| Stage of | 1.67 | 1.17-2.38 | 0.004 | 1.88 | 1.16-3.03 | 0.009 |
| disease | | | | | | |
| (ordinal) | | | | | | |
| Grading | 1.38 | 0.96-1.98 | 0.074 | 1.39 | 0.84-2.29 | 0.19 |
| (ordinal) | | | | | | |
| Residual tumor | 1.15 | 1.06-1.23 | <0.001 | 1.21 | 1.11-1.32 | <0.001 |
| (ordinal) | | | | | | |
| Histologic | 0.99 | 0.64-1.52 | 0.96 | 0.72 | 0.42-1.22 | 0.22 |
| type^{c} | | | | | | |
| Age | 1.02 | 0.99-1.04 | 0.06 | 1.02 | 0.99-1.05 | 0.074 |

^{a}Hazard ratio (HR) estimated from Cox proportional hazard regression model
^{b}Confidence interval of the estimated HR.
^{c}Serous vs. others

TABLE 3
Cox proportional hazard regression analysis for subgroups of patients

| | Progression-free survival | | | Overall survival | | |
|---|---|---|---|---|---|---|
| Variable | HR^{a} | 95% CI^{b} | P value | HR^{a} | 95% CI^{b} | P value |
| Tumor grade 1-2 | | | | | | |
| KLK9 unadjusted | 0.12 | 0.036-0.42 | <0.001 | 0.17 | 0.039-0.79 | 0.023 |
| KLK9 adjusted^{c} | 0.17 | 0.045-0.64 | 0.009 | 0.20 | 0.042-0.87 | 0.034 |
| Tumor grade 3 | | | | | | |
| KLK9 unadjusted | 0.73 | 0.45-1.18 | 0.21 | 0.84 | 0.43-1.63 | 0.61 |
| KLK9 adjusted^{c} | 0.75 | 0.46-1.25 | 0.27 | 0.81 | 0.44-1.52 | 0.52 |
| Stage I-II | | | | | | |
| KLK9 unadjusted | 0.097 | 0.012-0.77 | 0.028 | 0.20 | 0.63-2.06 | 0.61 |
| KLK9 adjusted^{d} | 0.10 | 0.011-0.89 | 0.039 | 0.90 | 0.17-4.35 | 0.88 |
| Stage III | | | | | | |
| KLK9 unadjusted | 0.65 | 0.41-1.032 | 0.068 | 0.71 | 0.40-1.26 | 0.24 |
| KLK9 adjusted^{d} | 0.75 | 0.46-1.21 | 0.23 | 0.81 | 0.44-1.49 | 0.51 |
| Optimal debulking success | | | | | | |
| KLK9 unadjusted | 0.32 | 0.12-0.83 | 0.018 | 0.80 | 0.21-2.98 | 0.74 |
| KLK9 adjusted^{e} | 0.31 | 0.12-0.86 | 0.025 | 0.68 | 0.16-2.91 | 0.61 |
| Suboptimal debulking success | | | | | | |
| KLK9 unadjusted | 0.83 | 0.50-1.38 | 0.48 | 0.82 | 0.43-1.56 | 0.55 |
| KLK9 adjusted^{e} | 0.69 | 0.40-1.19 | 0.18 | 0.67 | 0.34-1.31 | 0.24 |

^{a}Hazard ratio (HR) estimated from Cox proportional hazard regression model
^{b}Confidence interval of the estimated HR.
^{c}Multivariate models were adjusted for stage of disease, residual tumor, histologic type and age.
^{d}Multivariate models were adjusted for tumor grade, residual tumor, histologic type and age.
^{e}Multivariate models were adjusted for stage of disease, tumor grade, histologic type and age.

### FULL CITATIONS FOR REFERENCES REFERRED TO IN THE SPECIFICATION

1. Holschneider, C. H. and Berek, J. S. Ovarian cancer: epidemiology, biology, and prognostic factors, Semin Surg Oncol. 19: 3-10., 2000.
2. Greenlee, R. T., Murray, T., Bolden, S., and Wingo, P. A. Cancer statistics, 2000, CA Cancer J. Clin. 50: 7-33., 2000.
3. Zurawski, V. R, Jr., Orjaseter, H., Andersen, A., and Jellum, E. Elevated serum CA125 levels prior to diagnosis of ovarian neoplasia: relevance for early detection of ovarian cancer, Int J Cancer. 42: 677-80., 1988.
4. Vasilev, S. A., Schlaerth, J. B., Campeau, J., and Morrow, C. P. Serum CA125 levels in preoperative evaluation of pelvic masses, Obstet Gynecol. 71: 751-6., 1988.
5. Rubin, S. C., Hoskins, W. J., Hakes, T. B., Markman, M., Reichman, B. S., Chapman, D., and Lewis, J. L., Jr. Serum CA125 levels and surgical findings in patients undergoing secondary operations for epithelial ovarian cancer, Am J Obstet Gynecol. 160: 667-71., 1989.
6. Bridgewater, J. A., Nelstrop, A. E., Rustin, G. J., Gore, M. E., McGuire, W. P., and Hoskins, W. J. Comparison of standard and CA-125 response criteria in patients with epithelial ovarian cancer treated with platinum or paclitaxel, J Clin Oncol. 17: 501-8., 1999.
7. Meyer, T. and Rustin, G. J. Role of tumour markers in monitoring epithelial ovarian cancer, Br J Cancer. 82: 1535-8., 2000.
8. Chi, D. S., Venkatraman, E. S., Masson, V., and Hoslins, W. J. The ability of preoperative serum CA-125 to predict optimal primary tumor cytoreduction in stage III epithelial ovarian carcinoma, Gynecol Oncol. 77: 227-31., 2000.
9. Bast, R C., Jr., Klug, T. L., St John, E., Jenison, E., Niloff, J. M., Lazarus, H., Berkowitz, R. S., Leavitt, T., Griffiths, C. T., Parker, L., Zurawski, V. R., Jr., and Knapp, R. C. A radioimmunoassay using a monoclonal antibody to monitor the course of epithelial ovarian cancer, N Engl J. Med. 309: 883-7., 1983.
10. Berek, J. S. and Bast, R. C., Jr. Ovarian cancer screening. The use of serial complementary tumor markers to improve sensitivity and specificity for early detection, Cancer. 76: 2092-6., 1995.
11. Stenman, U. H., Alfthan, H., Vartiainen, J., and Lehtovirta, P. Markers supplementing CA125 in ovarian cancer, Ann Med. 27: 115-20., 1995.
12. Yousef, G. M., Obiezu, C. V., Luo, L. Y., Black, M. H., and Diamandis, E. P. Prostase/KLK-L1 is a new member of the human kallilrein gene family, is expressed in prostate and breast tissues, and is hormonally regulated, Cancer Res. 59: 4252-6, 1999.
13. Yousef, G. M. and Diamandis, E. P. The new kallikrein-like gene, KLK-L2. Molecular characterization, mapping, tissue expression, and hormonal regulation, J Biol. Chem. 274: 37511-6, 1999.
14. Yousef, G. M. and Diamandis, E. P. The expanded human kallikrein gene family: locus characterization and molecular cloning of a new member, KLK-L3 (KLK9), Genomics. 65: 184-194, 2000.
15. Yousef, G. M., Chang, A., and Diamandis, E. P. Identification and characterization of KLK-L4, a new kallikrein-like gene that appears to be down-regulated in breast cancer tissues, J Biol. Chem. 275: 11891-8, 2000.
16. Yousef, G. M., Magklara, A., and Diamandis, E. P. KLK12 is a novel serine protease and a new member of the human kallikrein gene family-differential expression in breast cancer, Genomics. 69: 331-41, 2000.
17. Yousef, G. M., Scorilas, A., Jung, K, Ashworth, L. K., and Diamandis, E. P. Molecular cloning of the human kallikrein 15 gene (KLK15). Up-regulation in prostate cancer, J Biol. Chem. 276: 53-61., 2001.
18. Yousef, G. M., Magklara, A., Chang, A., Jung, K, Katsaros, D., and Diamandis, E. P. Cloning of a new member of the human kallikrein gene family, KLK14, which is down-regulated in different malignancies, Cancer Res. 61: 3425-31., 2001.
19. Anisowicz, A., Sotiropoulou, G., Stenman, G., Mok, S. C., and Sager, R. A novel protease homolog differentially expressed in breast and ovarian cancer, Mol Med. 2: 624-36, 1996.
20. Liu, X. L., Wazer, D. E., Watanabe, K, and Band, V. Identification of a novel serine protease-like gene, the expression of which is down-regulated during breast cancer progression, Cancer Res. 56: 3371-9, 1996.
21. Yoshida, S., Taniguchi, M., Hirata, A., and Shiosaka, S. Sequence analysis and expression of human neuropsin cDNA and gene, Gene. 213: 9-16, 1998.
22. Yousef, G. M. and Diamandis, E. P. The new human tissue kallikrein gene family: structure, function and association to disease., Endo Rev. 22: 184-204, 2001.
23. Diamandis, E. P. Prostate-specific antigen-its usefulness in clinical medicine, Trends Endocrinol Metab. 9: 310-316, 1998.
24. Rittenhouse, H. G., Finlay, J. A., Mikolajczyk, S. D., and Partin, A. W. Human Kallikrein 2 (hK2) and prostate-specific antigen (PSA): two closely related, but distinct, kallikreins in the prostate, Crit Rev Clin Lab Sci. 35: 275-368, 1998.
25. Stenman, U. H. New ultrasensitive assays facilitate studies on the role of human glandular kallikrein (hK2) as a marker for prostatic disease [editorial; comment], Clin Chem. 45: 753-4, 1999.
26. Partin, A. W., Catalona, W. J., Finlay, J. A., Darte, C., Tindall, D. I., Young, C. Y., Klee, G. G., Chan, D. W., Rittenhouse, H. G., Wolfert, R. L., and Woodrum, D. L. Use of human glandular kallikrein 2 for the detection of prostate cancer: preliminary analysis, Urology. 54: 839-45, 1999.
27. Magklara, A., Scorilas, A., Catalona, W. J., and Diamandis, E. P. The combination of human glandular kallikrein and free prostate-specific antigen (PSA) enhances discrimination between prostate cancer and benign prostatic hyperplasia in patients with moderately increased total PSA, Clin Chem. 45: 1960-6, 1999.
28. Goyal, J., Smith, K. M., Cowan, J. M., Wazer, D. E., Lee, S. W., and Band, V. The role for NES1 serine protease as a novel tumor suppressor, Cancer Res. 58: 4782-6, 1998.
29. Tanimoto, H., Underwood, L. J., Shigemasa, K., Yan Yan, M. S., Clarke, J., Parmley, T. H., and O'Brien, T. J. The stratum corneum chymotryptic enzyme that mediates shedding and desquamation of skin cells is highly overexpressed in ovarian tumor cells, Cancer. 86: 2074-82, 1999.
30. Kim, H., Scorilas, A., Katsaros, D., Yousef, G. M., Massobrio, M., Fracchioli, S., Piccinno, R., Gordini, G., and Diamandis, E. P. Human kallikrein gene 5 (KLK5) expression is an indicator of poor prognosis in ovarian cancer, Br J Cancer. 84: 643-50., 2001.
31. Diamandis, E. P., Yousef, G. M., Soosaipillai, A. R., and Bunting, P. Human kallikrein 6 (zyme/protease M/neurosin): a new serum biomarker of ovarian carcinoma, Clin. Biochem. 33: 579-83, 2000.
32. Luo, L., Bunting, P., Scorilas, A., and Diamandis, E. P: Human kallikrein 10: a novel tumor marker for ovarian carcinoma?, Clin Chim Acta. 306: 111-8., 2001.
33. Diamandis, E. P., Yousef, G. M., Clements, J., Ashworth, L. K., Yoshida, S., Egelrud, T., Nelson, P. S., Shiosaka, S., Little, S., Lija, H., Stenman, U. H., Rittenhouse, H. G., and Wain, H. New nomenclature for the human tissue kallikrein gene family, Clin Chem. 46: 1855-8, 2000.
34. Underwood, L. J., Tanimoto, H, Wang, Y., Shigemasa, K., Parmley, T. H., and O'Brien, T. J. Cloning of tumor-associated differentially expressed gene-14, a novel serine protease overexpressed by ovarian carcinoma, Cancer Res. 59: 4435-9, 1999.
35. Magklara, A., Scorilas, A., Katsaros, D., Massobrio, M., Yousef, G. M., Fracchioli, S., Danese, S., and Diamandis, E. P. The Human KLK8 (Neuropsin/Ovasin) Gene: Identification of Two Novel Splice Variants and Its Prognostic Value in Ovarian Cancer. Clin Cancer Res. 7: 806-811., 2001.36.
36. Luo, L. Y., Grass, L., Howarth, D. J., Thibault, P., Ong, H., and Diamandis, E. P. Immunofluorometric assay of human kallikrein 10 and its identification in biological fluids and tissues, Clin Chem. 47: 237-46., 2001.
37. Serov, S. F. and Sorbin, L. H. Histological typing of ovarian tumors.: World Health Organization., 1973.
38. Pettersson, F. Annual report on the treatment in gynecological cancer., Vol. 22, p. 83-102. Stockholm: International Federation of Gynecology and Obstetrics, 1994.
39. Day, T. G., Jr., Gallager, H. S., and Rutledge, P. N. Epithelial carcinoma of the ovary:prognostic importance of histologic grade, Natl Cancer Inst Monogr. 42: 15-21., 1975.
40. Wittwer, C. T., Herrmann, M. G., Moss, A. A., and Rasmussen, R. P. Continuous fluorescence monitoring of rapid cycle DNA amplification, Biotechniques. 22: 1301, 134-8, 1997.
41. Bieche, L, Onody, P., Laurendeau, L, Olivi, M., Vidaud, D., Lidereau, R., and Vidaud, M. Real-time reverse transcription-PCR assay for future management of ERBB2-based clinical applications, Clin Chem. 45: 1148-56, 1999.
42. Bieche, I., Olivi, M., Champeme, M. H., Vidaud, D., Lidereau, R., and Vidaud, M. Novel approach to quantitative polymerase chain reaction using real-time detection: application to the detection of gene amplification in breast cancer, Int J Cancer. 78: 661-6, 1998.
43. Birren, B., Green, E. D., Klapholz, S., and al., e. Genome Analysis: A Laboratory Manual. Plainview, N.Y.: Cold Spring Harbor Laboratory Press, 1998.
44. Woo, T. H., Patel, B. K, Cinco, M., Smythe, L. D., Symonds, M. L., Norris, M. A., and Dohnt, M. F. Real-time homogeneous assay of rapid cycle polymerase chain reaction product for identification of Leptonema illini, Anal Biochem 259: 112-7, 1998.
45. Cox, D. R. Regression models and life tables., R. Stat. Soc. B. 34: 187-202, 1972.
46. Kaplan, E. L. and Meier, P. Nonparametric estimation from incomplete observations., J. Am. Atat. Assoc. 53: 457-481, 1958.
47. Mantel, N. Evaluation of survival data and two new rank order statistics arising in its consideration, Cancer Chemother Rep. 50: 163-70., 1966.
48. Menon, U. and Jacobs, I. J. Recent developments in ovarian cancer screening, Curr Opin Obstet Gynecol. 12: 39-42., 2000.
49. Jacobs, I. J., Skates, S. J., MacDonald, N., Menon, U., Rosenthal, A. N., Davies, A. P., Woolas, R., Jeyarajah, A. R., Sibley, K., Lowe, D. G., and Oram, D. H. Screening for ovarian cancer: a pilot randomised controlled trial, Lancet. 353: 1207-10., 1999.
50. Luo, L. Y., Katsaros, D., Scorilas, A., Fracchioli, S., Massobrio, M., Howarth, D., and Diamandis, E. P. Prognostic value of human kallikrein 10 expression in epithelial ovarian carcinoma, Clin. Cancer Res. In press:, 2001.
51. Fortier, A. H., Nelson, B. J., Grella, D. K., and Holaday, 3. W. Antiangiogenic activity of prostate-specific antigen, J Natl Cancer Inst. 91: 163-540, 1999.
52. Lai, L. C., Erbas, H., Lennard, T. W., and Peaston, R. T. Prostate-specific antigen in breast cyst fluid: possible role of prostate-specific antigen in hormone-dependent breast cancer, Int J Cancer. 66: 743-6, 1996.
53. Diamandis, E. P. Prostate-specific antigen: a cancer fighter and a valuable messenger?, Clin Chem. 46: 896-900, 2000.
54. Godwin, A. K., Testa, J. R., and Hamilton, T. C. The biology of ovarian cancer development, Cancer. 71: 530-6., 1993.
55. Langdon, S. P., Hawkes, M. M., Lawrie, S. S., Hawkins, R. A., Tesdale, A. L., Crew, A. J., Miller, W. R., and Smyth, J. F. Oestrogen receptor expression and the effects of oestrogen and tamoxifen on the growth of human ovarian carcinoma cell lines, Br J Cancer. 62: 213-6., 1990.
56. Murdoch, W. J. Perturbation of sheep ovarian surface epithelial cells by ovulation: evidence for roles of progesterone and poly(ADP-ribose) polymerase in the restoration of DNA integrity, J Endocrinol. 156: 503-8., 1998.
57. Munstedt, K, Steen, J., Knauf, A. G., Buch, T., von Georgi, R., and Franke, F. E. Steroid hormone receptors and long term survival in invasive ovarian cancer, Cancer. 89: 1783-91., 2000.
58. de la Cuesta, R., Maestro, M. L., Solana, J., Vidart, J. A., Escudero, M., Iglesias, E., and Valor, R. Tissue quantification of CA125 in epithelial ovarian cancer, Int J Biol Markers. 14: 106-14., 1999.

1. A method for detecting hK9 or KLK9 associated with ovarian cancer in a patient comprising:
(a) taking a sample from a patient;
(b) detecting or identifying in the sample hK9 or nucleic acid molecules encoding hK9; and
(c) comparing the detected amount with an amount detected for a standard.

2. A method for determining the presence or absence of ovarian cancer in a subject or a treatment of ovarian cancer in a subject comprising detecting a nucleic acid molecule encoding hK9 in a sample from the subject and relating the detected amount to the presence of ovarian cancer.

3. A method as claimed in claim 2 wherein the nucleic acid molecule detected is mRNA.

4. A method of claim 2 wherein the nucleic acid molecule is detected by
(a) contacting the sample with an oligonucleotide that hybridizes to the nucleic acid molecule encoding hK9; and
(b) detecting in the sample a level of polynucleotide that hybridizes to the nucleic acid molecule relative to a predetermined standard or cut-off value, and therefrom determining the presence or absence of ovarian cancer in the subject.

5. A method as claimed in claim 3 wherein the mRNA is detected using an amplification reaction.

6. A method as claimed in claim 5 wherein the amplification reaction is a polymerase chain reaction employing at least one oligonucleotide primer that hybridizes to a nucleic acid molecule that encodes hK9, or a complement of such nucleic acid molecule.

7. A method as claimed in claim 3 wherein the mRNA is detected using a hybridization technique employing an oligonucleotide probe that hybridizes to a nucleic acid molecule that encodes hK9, or a complement of such nucleic acid molecule.

8. A method as claimed in claim 3 wherein the mRNA is detected by (a) isolating mRNA from the sample and combining the mRNA with reagents to convert it to cDNA; (b) treating the converted cDNA with amplification reaction reagents and nucleic acid primers that hybridize to a nucleic acid molecule encoding hK9, to produce amplification products; (d) analyzing the amplification products to detect an amount of mRNA encoding hK9; and (e) comparing the amount of mRNA to an amount detected against a panel of expected values for normal and malignant tissue derived using similar nucleic acid primers.

9. A method for diagnosing and monitoring ovarian cancer in a subject comprising isolating nucleic acids, preferably mRNA, in a sample from the subject; and detecting KLK9 nucleic acids in the sample wherein the presence of increased levels of KLK9 nucleic acids in the sample compared to a standard or control is indicative of early disease stage, optimal debulking, and/or a positive prognosis.

10. A method of claim 9 wherein the standard or control is an amount determined for subjects with advanced stage ovarian cancer.

11. A method for determining the presence or absence of ovarian cancer in a subject comprising detecting hK9 in a sample from the subject and relating the detected amount to the presence of ovarian cancer.

12. A method as claimed in claim 11 comprising
(a) contacting a biological sample obtained from a subject with a binding agent that specifically binds to hK9 or a part thereof; and
(b) detecting in the sample an amount of hK9 that binds to the binding agent, relative to a predetermined standard or cut-off value, and therefrom determining the presence or absence of ovarian cancer in the subject.

13. A method as claimed in claim 12 wherein the binding agent is an antibody.

14. A method as claimed in claim 1 wherein the sample is obtained from tissues, extracts, cell cultures, cell lysates, or physiological fluids.

15. A method as claimed in claim 14 wherein the sample is obtained from a tumor tisue.

16. A method as claimed claim 1 which further comprises detecting one or more of human stratum corneum chymotryptic enzyme (HSCCE), haptoglobin alpha, osteopontin, kallikrein 4, kallikrein 5, kallikrein 6, kallikrein 8, kallikrein 10, kallikrein 11, CA125, CA15-3, CA19-9, CA72-4, OVX1, lysophosphatidic acid (LPA), creatin-kinase BB, or carcinoembryonic antigen (CEA).

17. A diagnostic composition comprising an agent that binds to hK9 or hybridizes to a nucleic acid molecule encoding hK9.

18. An in vivo method for imaging ovarian cancer comprising:
(a) injecting a patient with an agent that binds to hK9, the agent carrying a label for imaging the ovarian cancer;
(b) allowing the agent to incubate in vivo and bind to hK9 associated with the ovarian cancer; and
(c) detecting the presence of the label localized to the ovarian cancer.

19. A method as claimed in claim 18 wherein the agent is an antibody that specifically reacts with hK9.

20. A method as claimed in claim 18 wherein the label is a radiolabel, fluorescent label, nuclear magnetic resonance active label, positron emitting isotope detectable by a positron emission tomography (“PET”) scanner, chemiluminescer, or enzymatic marker.

21. A kit for carrying out a method as claimed in claim 1.

22. A kit for determining the presence of ovarian cancer in a subject, comprising a known amount of a binding agent that specifically binds to kallikrein 9 wherein the binding agent comprises a detectable substance, or it binds directly or indirectly to a detectable substance.

23. A kit for determining the presence of ovarian cancer in a subject, comprising a known amount of an oligonucleotide that hybridizes to a nulceic acid molecule encoding hK9 wherein the oligonucleotide is directly or indirectly labeled with a detectable substance.
